(19) United States
(12) Patent Application Publication
(10) Pub. No.: US 2004/0101840 A1
(43) Pub. Date: May 27, 2004

(54) Identification, cloning and characterization of a novel pc-1 gene differentially expressed in prostate cells and tissues

(76) Inventors: Leland W.K. Chung, Atlanta, GA (US); Jian-Guang Zhou, Beijing (CN)

Correspondence Address: JONES DAY, 222 EAST 41ST STREET, NEW YORK, NY, 10017, US

(21) Appl. No.: 10/258,966

(22) PCT Filed: Apr. 27, 2001
(86) PCT No.: PCT/US01/13666

(51) Int. Cl. C12Q 1/68; G01N 33/574

(52) U.S. Cl. 435/6; 435/7.23

(57) ABSTRACT

The present invention relates to the isolation, cloning, and characterization of a prostate-specific PC-1 gene differentially expressed by prostate cells and tissues. The present invention additionally relates to replication-competent adenovirus vectors which are specific for cells which allow a PC-1 transcriptional regulatory sequence to function, such as prostate cancer cells, prostate stromal cells, breast cancer cells, renal cells, bladder cells, and cells of the endometrium, and methods of use of such viruses are provided. These viruses comprise an adenoviral gene under control of a PC-1 transcriptional regulatory sequence. Such replication-competent adenovirus vectors are useful in the treatment of prostate cancer, brain cancer, ovarian cancer, thyroid cancer, tumors, renal carcinoma, bladder cancer, lung cancer, or breast cancer.

This application claims the benefit under 35 U.S.C. section 119(e) of co-pending U.S. provisional application 60/200,526, filed Apr. 27, 2000, the entire text of which is herein incorporated by reference without disclaimer.

### 1 INTRODUCTION

The present invention describes the isolation, cloning, and characterization of a novel prostate-specific gene, PC-1, in human prostate cells and tissues. Based upon the knowledge of PC-1 gene regulation by androgen and expression profiles in human tissues, the promoter of the PC-1 gene can be used for the delivery of nucleic acids specifically into prostate cells. The PC-1 promoter can also be expected to deliver specific nucleic acid sequences into prostate cells for transgenic studies. Moreover, antibodies which specifically recognize the expression products of this gene can be used for diagnostic, prognostic, and treatment of prostatic-related diseases.

### 2 BACKGROUND

Prostate cancer is the most commonly diagnosed malignancy and the second leading cause of cancer death among U.S. males. The diagnosis of prostate cancer was greatly helped by the discovery of a prostate-specific protein called prostate specific antigen (PSA). Following the discovery of the PSA gene, much effort in recent years has focused on the discovery of other human kallikreins, which consist of at least three members: PSA, glandular kallikrein (hGK-1), and pancreatic/renal kallikrein (hPRK). Other than the PSA gene, recent studies have also established several other human prostate-specific genes, such as prostate specific membrane antigen (PSMA) and prostate stem cell antigen (PSCA). These discoveries of prostate-specific genes could have great utility in the expansion of the diagnosis, prognosis, and treatment of prostate cancer and improve the possibility of delivering therapeutic genes specifically to diseased prostatic epithelial cells. For these reasons, a number of laboratories have focused on the isolation of prostate-specific genes either from rat, mouse, or human sources using a number of molecular biologic techniques. In recent years, because of improvements in new gene discoveries using microarray techniques, newer genes and ESTs are being discovered at a rapid rate.

In the present study, the inventors hypothesized that during prostate cancer progression, a number of genetic aberrations may have occurred during disease progression. Specifically, the inventors applied this hypothesis to well-established lineage-derived human prostate cancer cell lines, the parental LNCaP and the derived C4, C4-2, and C4-2B cell lines, as a model to assess the molecular basis of prostate cancer progression.

In previous studies, the inventors established that specific genetic alterations including regions of gene loss and gain occurred in this cell model of prostate cancer progression. Moreover, the inventors obtained evidence that the expression of genes in this cell model of prostate cancer progression mimicked clinical prostate cancer. For example, the inventors have found that a number of these noncollagenous bone matrix proteins, such as osteocalcin, osteopontin, and bone sialoprotein mimicked the clinical observation where the expression of these bone matrix proteins may be enhanced. Using this cell model of prostate carcinogenesis, the inventors have cloned a new member of the MAGE/GAGE melanoma-related antigen called PAGE-1 (to indicate a prostate cancer-specific antigen) which was found to be increased in Al and metastatic prostate cancer cells.

### 3 SUMMARY OF INVENTION

The present invention relates to the use of a cDNA microarray method which allowed for the identification of a series of genes that are up- or down-regulated in C4-2 cells, an AI (androgen independent) and metastatic human prostate cancer cell line derived from a parental LNCaP cells (a marginal tumorigenic androgen-dependent (AD), and nonmetastatic human prostate cancer cell line). More specifically, the present invention relates to the isolation, cloning, and characterization of a prostate-specific PC-1 gene differentially expressed by prostate cells and tissues through the use of cDNA microarray technique. This gene was found to be differentially expressed by human prostate with very low levels of expression detected in the human colon and kidney.

Using a sensitive RT-PCR technique, the PC-1 gene was also found to be expressed by a number of tumor cell lines including prostate cancer, breast cancer, renal cell carcinoma, bladder cancer, and endometrium carcinoma. PC-1 gene expression was further demonstrated to be positively regulated by androgen, with levels of gene expression enhanced in lineage-derived human prostate cancer cell lines upon androgen-independent (Al) progression. The PC-1 gene was found to be expressed in nearly 100% of human normal, BPH, and cancer tissues. The PC-1 gene was mapped to chromosome 81q21, a region frequently amplified in human prostate cancer. Results from protein alignments of the PC-1 gene with other known genes in the gene bank revealed marked homology with known genes, D52/N8, which shared identical nucleotide sequences from 462-2,552 base pairs. The 5′-end of PC-1 cDNA sequence, from 1-461 base pairs, however, was found to share no sequence homology with known genes in the GenBank. Unlike D52/N8 genes, the PC-1 gene is unique in that it appears to have a different tissue expression profile and its expression is regulated by androgen in prostate cancer cell lines.

The present invention additionally relates to compositions related to novel viral vectors which can be used as therapeutic agents for treating metastatic cancers, including, without limitation, prostate cancer, brain cancer, ovarian cancer, thyroid cancer, tumors, lung cancer, renal carcinoma, bladder cancer, breast cancer and those diseases involving calcification, including, without limitation, benign prostate hyperplasia (BPH). The present invention further relates to novel methods for using the therapeutic compositions.

The invention is also based, in part, on the fact that adenoviral vectors constructed with a PC-1 transcriptional regulatory sequence described herein are capable of selectively driving expression of an adenovirus gene essential for replication in a tissue specific and tumor-restrictive manner. The invention is further based, in part, on the discovery that such adenoviral vectors can be used as therapeutic agents for treating prostate cancer. Thus, due to the tissue-specificity and tumor-restrictiveness of the PC-1 transcriptional regulatory sequence used with the adenoviral vectors, the adenovirus can be administered in a tumor-restrictive and tissue-specific manner, with the use of a PC-1 transcriptional regulatory sequence which allows for tissue specific expression of the adenovirus gene essential for replication and/or heterologous nucleotide sequence. An example of such a PC-1 transcriptional regulatory sequence is the PC-1 promoter which is activated only within cells of prostatic lineage. Thus, an adenovirus vector constructed with an essential gene under the control of an PC-1 transcriptional regulatory sequence can be expressed effectively and specifically in targeted tumor cells and tissues, thereby minimizing the side effects of expression of the adenovirus vector in non-prostatic cells.

In addition, due to the tissue specificity of the PC-1 transcriptional regulatory sequence used with the adenoviral vectors, the viral vectors of the present invention are effective therapeutic agents not only when administered via direct application, such as by injection, but also when administered systemically to the body via intravenous administration, oral administration or the like, because gene expression will be limited and localized to specific, prostatic cell and disease tissues.

In one embodiment, the invention provides an adenovirus vector comprising an adenovirus with an essential gene under transcriptional control of a PC-1 transcriptional regulatory sequence. The PC-1 transcriptional regulatory sequence is capable of mediating gene expression specific to cells which allow an PC-1 transcriptional regulatory sequence to function, such as for example, and without limitation, prostate cancer cells, prostate stromal cells, breast cancer cells, renal cells, bladder cells, and cells of the endometrium. The PC-1 transcriptional regulatory sequence can comprise a promoter and/or enhancer or enhancer-like sequence from an PC-1 gene, provided that the PC-1 transcriptional regulatory sequence is capable of mediating gene expression specific to cells expressing PC-1. In one embodiment, a PC-1 transcriptional regulatory sequence comprises a promoter from a PC-1 gene. In one embodiment, a PC-1 transcriptional regulatory sequence comprises an enhancer or enhancer-like sequence from a PC-1 gene. In one embodiment, a PC-1 transcriptional regulatory sequence comprises a promoter from a PC-1 gene and an enhancer or enhancer-like sequence from a PC-1 gene. In one embodiment, the PC-1 transcriptional regulatory sequence is transcriptionally active in cells which allow a PC-1 transcriptional regulatory sequence to function, such as cells expressing PC-1.

In certain embodiments, an PC-1 transcriptional regulatory sequence comprises the 1-322 bp nucleotide sequence of SEQ ID NO:1 as shown in FIG. 2. In certain embodiments, a PC-1 transcriptional regulatory sequence comprises a portion of SEQ ID NO:1 capable of mediating cell-specific transcription in PC-1-producing cells such as for example, without limitation, prostate cancer cells, prostate stromal cells, breast cancer cells, renal cells, bladder cells, and cells of the endometrium. In another embodiment, a PC-1 transcriptional regulatory sequence comprises the sequence from about −290 to about +30 relative to the transcriptional start site of the PC-1 gene of SEQ ID NO:1. In another embodiment, a PC-1 transcriptional regulatory sequence comprises the sequence from about −250 to about +30 relative to the transcriptional start site of the PC-1 gene of SEQ ID NO:1. In another embodiment, a PC-1 transcriptional regulatory sequence comprises the sequence to about −236 to about −223 and/or the sequence to about −140 to about −117 relative to the transcriptional start site of the PC-1 gene of SEQ ID NO:1, combined with a non-PC-1 promoter. In yet another embodiment, a PC-1 transcriptional regulatory sequence comprises the nucleotide sequence from nucleotides about 1 to about 100, about 1 to about 150, about 1 to about 200, about 1 to about 250, about 1 to about 300, and about 1-322, respectively, of SEQ ID NO:1. In each embodiment, a PC-1 transcriptional regulatory sequence is defined as a transcriptional regulatory sequence or transcriptional regulatory sequence capable of effecting transcription in a cell, which allows a PC-1 transcriptional regulatory sequence to function, such as a cell expressing PC-1, such as for example, without limitation, prostate cancer cells, prostate stromal cells, breast cancer cells, renal cells, bladder cells, and cells of the endometrium.

In some embodiments, the PC-1 transcriptional regulatory sequence is human, mouse, or rat in origin. In some embodiments, the mouse or rat PC-1 transcriptional regulatory sequence is capable of mediating prostate-specific gene expression in humans.

In some embodiments, the adenovirus gene under control of an PC-1 transcriptional regulatory sequence contributes to cytotoxicity (directly or indirectly), such as a gene essential for viral replication. In one embodiment, the adenovirus gene is an early gene. In another embodiment, the early gene is E1A. In another embodiment, the early gene is E1B. In yet another embodiment, both E1A and E1B are under transcriptional control of an PC-1 transcriptional regulatory sequence. In other embodiments, the adenovirus gene essential for replication is a late gene. In various embodiments, the additional late gene is L1, L2, L3, L4, or L5.

In another embodiment, the adenovirus vector comprising an adenovirus gene under transcriptional control of a PC-1 transcriptional regulatory sequence further comprises at least one additional adenovirus gene under transcriptional control of at least one additional PC-1-specific transcriptional regulatory sequence. In one embodiment, a composition comprises this adenovirus. In one embodiment, this composition further comprises a pharmaceutically acceptable excipient. In one embodiment, the at least one additional PC-1-specific transcriptional regulatory sequence is a second PC-1 transcriptional regulatory sequence. In one embodiment, the at least one additional PC-1 transcriptional regulatory sequence can have a sequence different from that of the first PC-1 transcriptional regulatory sequence. In one embodiment, the at least one additional PC-1-specific transcriptional regulatory sequence comprises a PC-1 transcriptional regulatory sequence.

In other embodiments, the adenovirus vector can further comprise a heterologous gene or transgene, wherein said heterologous gene or transgene is under the transcriptional control of a PC-1 transcriptional regulatory sequence. In one embodiment, the heterologous gene is a reporter gene such as for example, and without limitation, the luciferase reporter gene or beta-galactosidase reporter gene. In one embodiment, the heterologous gene is conditionally required for cell survival. In some embodiments, the transgene is a cytotoxic gene.

In another embodiment, a method of treating metastatic cancer in an individual is provided, the method comprising the step of administering to the individual an effective amount of an adenovirus vector in which an adenovirus gene is under transcriptional control of a PC-1 transcriptional regulatory sequence, wherein the metastatic cancer is prostate cancer. In another embodiment, a method of treating metastatic cancer in an individual is provided, the method comprising the step of administering to the individual an effective amount of an adenovirus vector in which an adenovirus gene is under transcriptional control of a PC-1 transcriptional regulatory sequence, wherein the metastatic cancer is prostate cancer.

In one embodiment, the adenovirus gene is essential for viral replication. In one embodiment, the adenovirus gene is an early gene. In one embodiment, the adenovirus gene is E1A. In one embodiment, the adenovirus gene is E1B. In one embodiment, the PC-1 transcriptional regulatory sequence comprises an enhancer or enhancer-like sequence from an PC-1 gene. In one embodiment, the PC-1 transcriptional regulatory sequence comprises a promoter from a PC-1 gene. In one embodiment, the PC-1 transcriptional regulatory sequence comprises a promoter from a PC-1 gene and an enhancer or enhancer-like sequence from a PC-1 gene. In one embodiment, the adenovirus further comprises an additional adenovirus gene under transcriptional control of at least one additional transcriptional regulatory sequence. In one embodiment, the second transcriptional regulatory sequence comprises a PC-1 transcriptional regulatory sequence. In one embodiment, the additional adenovirus gene is essential for viral replication. In one embodiment, the additional adenovirus gene is an early gene. In one embodiment, the additional adenovirus gene is E1A. In one embodiment, the additional adenovirus early gene is E1B. In one embodiment, the additional adenovirus gene is a late gene. In various embodiments, the late gene can be L1, L2, L3, L4, or L5.

In another aspect, the invention provides a host cell transformed with any adenovirus vector(s) described herein.

In another aspect, the invention provides a composition comprising an adenovirus vector comprising an adenovirus gene under transcriptional control of an PC-1 transcriptional regulatory sequence. In one embodiment, the composition further comprises a pharmaceutically acceptable excipient.

In another aspect, the invention provides kits which contain an adenoviral vector(s) described herein.

In another aspect, a method is provided for propagating an adenovirus vector specific for cells which allow an PC-1 transcriptional regulatory sequence to function, such cells including, for example, prostate cancer cells, prostate stromal cells, breast cancer cells, renal cells, bladder cells, and cells of the endometrium, said method comprising infecting such cells which allow an PC-1 transcriptional regulatory sequence to function with any of the adenovirus vector(s) described herein, whereby said adenovirus vector is propagated.

In another aspect, a method for modifying the genotype of a target cell is provided, the method comprising contacting a cell which allows a PC-1 transcriptional regulatory sequence to function, such cells including, for example, prostate cancer cells, prostate stromal cells, breast cancer cells, renal cells, bladder cells, and cells of the endometrium, with any adenovirus described herein, wherein the adenovirus enters the cell.

In another aspect, methods are provided for detecting cells expressing PC-1 in a biological sample, comprising contacting cells of a biological sample with an adenovirus vector(s) described herein, and detecting replication of the adenovirus vector, if any.

In one embodiment, a method is provided for detecting cells which allow a PC-1 transcriptional regulatory sequence to function, for example, prostate cancer cells, prostate stromal cells, breast cancer cells, renal cells, bladder cells, and cells of the endometrium, in a biological sample, the method comprising the steps of: contacting a biological sample with an adenovirus vector comprising an essential adenoviral early or late gene under transcriptional control of a PC-1 transcriptional regulatory sequence, under conditions suitable for PC-1 transcriptional regulatory sequence-mediated gene expression in cells which allow a PC-1 transcriptional regulatory sequence to function; and determining if the PC-1 transcriptional regulatory sequence mediates gene expression in the biological sample, where PC-1 transcriptional regulatory sequence-mediated gene expression is indicative of the presence of cells which allow a PC-1 transcriptional regulatory sequence to function. In one embodiment, the gene is a heterologous (non-adenovirus gene). In one embodiment, the heterologous gene is a reporter gene, and production of the product of the reporter gene is detected.

In another embodiment, a method is provided for conferring selective toxicity or cytotoxicity on a target cell, said method comprising contacting a target cell which allows a PC-1 transcriptional regulatory sequence to function, for example, without limitation, in prostate cancer cells, prostate stromal cells, breast cancer cells, renal cells, bladder cells, and cells of the endometrium, with any adenovirus disclosed herein, wherein the adenovirus enters the cell.

In yet another embodiment, an adenovirus is provided which comprises a heterologous gene under transcriptional control of a PC-1 transcriptional regulatory sequence. In one embodiment, the heterologous gene is a reporter gene. In one embodiment, the heterologous gene is conditionally required for cell survival. In one embodiment, a method is provided for detecting cells which allow a PC-1 transcriptional regulatory sequence to function, such as, for example, without limitation, prostate cancer cells, prostate stromal cells, breast cancer cells, renal cells, bladder cells, and cells of the endometrium, in a sample comprising the steps of: contacting a biological sample with an adenovirus vector comprising a gene under transcriptional control of a PC-1 transcriptional regulatory sequence, under conditions suitable for PC-1 transcriptional regulatory sequence-mediated gene expression in cells which allow an PC-1 transcriptional regulatory sequence to function; and determining if PC-1 transcriptional regulatory sequence mediates gene expression in the biological sample, where PC-1 transcriptional regulatory sequence-medicated gene expression is indicative of the presence of cells expressing PC-1.

As described in more detail herein, an PC-1 transcriptional regulatory sequence can comprise any number of configurations, including, but not limited to, a PC-1 promoter; a PC-1 enhancer or PC-1 enhancer-like sequence; a PC-1 silencer; a PC-1 promoter and a PC-1 enhancer or PC-1 enhancer-like sequence; a PC-1 promoter and a non-PC-1 (heterologous) enhancer; a non-PC-1 (heterologous) promoter and a PC-1 enhancer or PC-1 enhancer-like sequence; a non-PC-1 promoter and multiple copies of enhancers; and multimers of the foregoing. Methods are described herein for measuring the activity of a PC-1 transcriptional regulatory sequence and thus for determining whether a given cell allows an PC-1 transcriptional regulatory sequence to function. The promoter and enhancer or PC-1 enhancer-like sequence of a PC-1 transcriptional regulatory sequence may be in any orientation and/or distance from the coding sequence of interest, and may comprise multimers of the foregoing, as long as the desired PC-1 cell-specific transcriptional activity is obtained. Transcriptional activation can be measured in a number of ways known in the art (and as described in more detail below), but is generally measured by detection and/or quantitation of mRNA or the protein product of the coding sequence under control of (i.e., operatively linked to) a PC-1 transcriptional regulatory sequence. As discussed herein, a PC-1 transcriptional regulatory sequence can be of varying lengths, and of varying sequence composition.

By “transcriptional activation” or an “increase in transcription”, it is intended that transcription will be increased above basal levels in the target cell (i.e. cells that allow a PC-1 transcriptional regulatory sequence to function, such as, for example, without limitation, prostate cancer cells, prostate stromal cells, breast cancer cells, renal cells, bladder cells, and cells of the endometrium by at least about 20-fold, more preferably at least about 50-fold, more preferably at least about 100-fold, even more preferably at least about 200-fold, even more preferably at least about 400- to about 500-fold, even more preferably, at least about 1000-fold. Basal levels are generally the level of activity, if any, in a non-PC-1-producing cell, or the level of activity (if any) of a reporter construct lacking an PC-1 transcriptional regulatory sequence as tested in a PC-1-producing cell. Optionally, a transcriptional terminator or transcriptional “silencer” can be placed upstream of the PC-1 transcriptional regulatory sequence, thereby preventing unwanted read-through transcription of the coding segment under transcriptional control of the PC-1 transcriptional regulatory sequence. Also, optionally, the endogenous promoter of the coding segment to be placed under transcriptional control of the PC-1 transcriptional regulatory sequence can be deleted.

Another embodiment of the invention is an adenovirus which replicates preferentially in mammalian cells expressing osteocaclin.

### 4 BRIEF SUMMARY OF THE DRAWINGS

FIG. 1 represents a Northern blot analysis of nine genes expressed by AD parental LNCaP and its Al C4-2 cell lines. Of a total of 1,600 distinct cDNAs represented on the microarray, 16 cDNAs were identified with 2-6 fold higher expression in C4-2 than LNCaP cells. Based on the Northern blot analysis, the 5B-10 gene or the PC-1 gene was found to be markedly elevated in C4-2 cells (5 fold increase in expression in C4-2 than parental LNCaP cells). 900 bp of the 3′-end of the SB-10 cDNA was sequenced and confirmed to have sequence homology with two known genes, D52 (gene bank accession #4827037) and N8 (gene bank accession #582081).

FIG. 2 represents the complete cDNA nucleotide sequence of the novel PC-1 gene. Also depicted is the amino acid sequence of PC-1. Full length PC-1 cDNA was obtained by a 5′-RACE and 3′-RACE using the Clontech Smart TM RACE cDNA amplification kit (Clontech, Palo Alto, Calif.) according to the manufacturer's instructions.

FIG. 3 represents a complete alignment of the PC-1 gene with other known genes, such as D52/N8, N8L, CSPP28, R10, and mD52. Significant homology was noted between the PC-1 gene and the other genes, however the 5′-coding region of the PC-1 gene is distinctly different from other genes based on GenBank search. The PC-1 gene is a novel gene that belongs to a member of the D52/N8 family.

FIG. 4 shows a RT-PCR CaP and Southern blot analyses of the PC-1 gene expressed in normal human prostate tissues with minor expression in normal human colon and kidney tissues. This result was also confirmed by Northern blot analysis, where the PC-1 gene is expressed predominantly in normal human prostate tissues with minor expression in normal human colon tissues only.

FIG. 5 indicates that the PC-1 gene expression is regulated in LNCaP and C4-2 cells by androgen. As indicated in this figure, steady-state levels of PC-1 gene expression is increased in C4-2 cells by exposing cells to a synthetic androgen, R1881, at 0.1 nM but a 10 fold higher concentration of R1881, 1 nM, is required to increase PC-1 gene expression in LNCaP cells. The increased expression of the PC-1 gene can be blocked by the coadministration of an antiandrogen, Casodex (10 nM). In sharp contrast, the expression of the family members of PC-1 gene, D52/N8, was not regulated by androgen nor was its expression inhibited by the coadministration of R1881 and Casodex.

FIG. 6 represents PC-1 gene expression in different human cell lines. It is indicated that the PC-1 gene is preferentially expressed in prostate cancer cell lines, including LNCaP and its derivative C4, C4-2, C4-2B cell lines, and ARCaP, DU145, and PC-3 cells. In addition, a human breast cancer cell line (MCF-7) human renal cell carcinoma (RCC-38), human bladder transitional cell carcinoma cell line (WH), and a human endometrium cell line (HELA), and a peripheral zone-derived prostate cancer cell line (9069 RP-5) were found to express PC-1 gene based on RT-PCR. A human osteosarcoma cell line (MG-63), a renal cell carcinoma cell line (RCC-42), and a clinical prostate cancer derived cell line (9069 E) were found to be absent in PC-1 gene expression.

FIG. 7 shows that the PC-1 gene is distinct form D52/N8 based on Northern blot analysis in several human prostate cancer cell lines. As shown in this figure, Probe B (1-461 bp) was used to recognize specifically PC-1 gene hybridizization against a 2.6 Kb PC-1 gene; however, no hybridization occurred against the D52/N8 gene. In contrast, as expected, Probe A which recognizes a common sequence between the PC-1 and D52/N8 gene (probe designed to recognized 461-2,552 bp) hybridized to both the PC-1 and D52/N8 gene.

FIG. 8 indicates the expression of the PC-1 gene in normal human prostate, benign prostatic hyperplasia (BPH) prostate tissues, and prostate cancer. As shown in the top panel, prostate cancer tissues prevalently expressed the PC-1 gene. Similarly, in the bottom panel B, PC-1 gene is prevalently expressed in both normal human prostate and BPH tissues.

FIG. 9 shows the results of the northern hybridization which indicate that the human PC1 gene is predominantly expressed in prostate tissue.

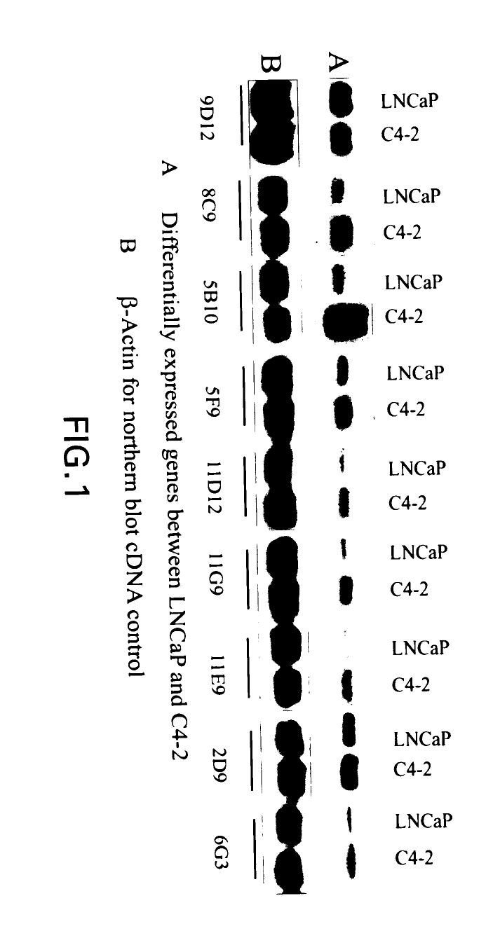

### DEFINITIONS

For convenience, the meaning of certain terms and phrases employed in the specification, examples, and appended claims are provided below.

The term “tissue-specific” is intended to mean that the transcriptional regulatory sequence to which the gene essential for viral replication is operably linked functions in that tissue so that replication proceeds in that tissue.

The term “transcriptional regulatory sequence” is used according to its art-recognized meaning. It is intended to mean any DNA sequence which can, by virtue of its sequence, cause the linked gene to be either up- or down-regulated in a particular cell. In one embodiment of the present invention, the native transcriptional regulatory sequence is completely deleted from the vector and replaced with a heterologous transcriptional regulatory sequence. The transcriptional regulatory sequence may be adjacent to the coding region for the gene that is essential for replication, or may be removed from it. Accordingly, in the case of a promoter, the promoter will generally be adjacent to the coding region. In the case of an enhancer or enhancer-like sequence, however, an enhancer or enhancer-like sequence can be found at some distance from the coding region such that there is an intervening DNA sequence between the enhancer or enhancer-like sequence and the coding region. In some cases, the native transcriptional regulatory sequence remains on the vector but is non-functional with respect to transcription of the gene essential for replication. In some cases, the native transcriptional regulatory sequence remains on the vector and is augmented by placement of the tissue-specific tumor-restrictive transcriptional regulatory sequence to which the gene essential for viral replication is operably linked.

An “adenovirus vector” or “adenoviral vector” (used interchangeably) is a term well understood in the art and generally comprises a polynucleotide (defined herein) comprising all or a portion of an adenovirus genome. For purposes of the present invention, an adenovirus vector contains an PC-1 transcriptional regulatory sequence operably linked to a polynucleotide. The operably linked polynucleotide can be adenoviral or heterologous. An adenoviral vector construct of the present invention can be in any of several forms, including, but not limited to, naked DNA, DNA encapsulated in an adenovirus coat, DNA encapsulated in liposomes, DNA complexed with polylysine, complexed with synthetic polycationic molecules, conjugated with transferrin, and complexed with compounds such as PEG to immunologically “mask” the molecule and/or increase half-life, or conjugated to a non-viral protein. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides. For purposes of this invention, adenovirus vectors are replication-competent in a target cell such as a tumor cell.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxy-nucleoside phosphoramidate (P—NH₂) or a mixed phosphoramidate-phosphodiester oligomer. Peyrottes et al. (1996) Nucleic Acids Res. 24:1841-8; Chaturvedi et al. (1996) Nucleic Acids Res. 24:2318-23; Schultz et al. (1996) Nucleic Acids Res. 24:2966-73. A phosphorothiate linkage can be used in place of a phosphodiester linkage. Braun et al. (1988) J. Immunol. 141:2084-9; Latimer et al. (1995) Mol. Immunol. 32:1057-1064. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

A polynucleotide or polynucleotide region has a certain percentage (for example, 80%, 85%, 90%, 95%, 98%, or 99%) of “sequence identity” to another sequence means that, when aligned, that percentage of bases are the same in comparing the two sequences. This alignment and the percent homology or sequence identity can be determined using software programs known in the art, for example, those described in Current Protocols in Molecular Biology (Ausubel et al., eds., 1987), Supp. 30, section 7.7.18, Table 7.7.1. A preferred alignment program is ALIGN Plus (Scientific and Educational Software, Pennsylvania).

As used herein, “a cell which allows a PC-1 transcriptional regulatory sequence to function”, a cell in which the function of a PC-1 transcriptional regulatory sequence is “sufficiently preserved”, “a cell in which a PC-1 transcriptional regulatory sequence functions” is a cell in which a PC-1 transcriptional regulatory sequence, when operably linked to, for example, a reporter gene, increases expression of the reporter gene at least about 20-fold, more preferably at least about 50-fold, more preferably at least about 100-fold, more preferably at least about 200-fold, even more preferably at least about 400- to 500-fold, even more preferably at least about 1000-fold, when compared to the expression of the same reporter gene when not operably linked to the PC-1 transcriptional regulatory sequence. Methods for measuring levels (whether relative or absolute) of expression are known in the art and are described herein.

“Under transcriptional control” is a term well-understood in the art and indicates that transcription of a polynucleotide sequence, usually a DNA sequence, depends on its being operably (operatively) linked to an element or transcriptional regulatory sequence which contributes to the initiation of, or promotes, transcription. As noted below, “operably linked” refers to a juxtaposition wherein the elements transcriptional regulatory sequences are in an arrangement allowing them to function.

As used herein, “cytotoxicity” is a term well understood in the art and refers to a state in which one or more of a cell's usual biochemical or biological functions are aberrantly compromised (i.e., inhibited or elevated). These activities include, but are not limited to metabolism; cellular replication; DNA replication; transcription; translation; and uptake of molecules. “Cytotoxicity” includes cell death and/or cytolysis. Assays are known in the art which indicate cytotoxicity, such as dye exclusion, ³H-thymidine uptake, and plaque assays. The term “selective cytotoxicity”, as used herein, refers to the cytotoxicity conferred by an adenovirus vector of the present invention on a cell which allows a PC-1 transcriptional regulatory sequence to function when compared to the cytotoxicity conferred by the adenovirus on a cell which does not allows a PC-1 transcriptional regulatory sequence to function. Such cytotoxicity may be measured, for example, by plaque assays, reduction or stabilization in size of a tumor comprising target cells, or the reduction or stabilization of serum levels of a marker characteristic of the tumor cells or a tissue-specific marker, e.g., a cancer marker such as prostate specific antigen.

“Replication” and “propagation” are used interchangeably and refer to the ability of a adenovirus vector of the invention to reproduce or proliferate. This term is well understood in the art. For purposes of this invention, replication involves production of adenovirus proteins and is generally directed to reproduction of adenovirus. Replication can be measured using assays standard in the art and described herein, such as a burst assay or plaque assay. “Replication” and “propagation” include any activity directly or indirectly involved in the process of virus manufacture, including, but not limited to, viral gene expression; production of viral proteins, nucleic acids or other components; packaging of viral components into complete viruses; and cell lysis.

The term “heterologous” means a DNA sequence not found in the native vector genome. With respect to a “heterologous transcriptional regulatory sequence”, “heterologous” indicates that the transcriptional regulatory sequence is not naturally ligated to the DNA sequence for the gene essential for replication of the vector.

A “heterologous gene” or “transgene” is any gene that is not present in wild-type adenovirus. Preferably, the transgene will also not be expressed or present in the target cell prior to introduction by the adenovirus vector. Examples of preferred transgenes are provided below.

The term “promoter” is used according to its art-recognized meaning. It is intended to mean the DNA region, usually upstream to the coding sequence of a gene or operon, which binds RNA polymerase and directs the enzyme to the correct transcriptional start site.

The term “enhancer” is used according to its art-recognized meaning. It is intended to mean a sequence found in eukaryotes and certain eukaryotic viruses which can increase transcription from a gene when located (in either orientation) up to several kilobases from the gene being studied. These sequences usually act as enhancers when on the 5′ side (upstream) of the gene in question. However, some enhancers are active when placed on the 3′ side (downstream) of the gene. The enhancer may also be an enhancer-like sequence.

The term “silencer,” used in its art-recognized sense, means a sequence found in eucaryotic viruses and eucaryotes which can decrease or silence transcription of a gene when located within several kilobases of that gene.

A “heterologous” promoter or enhancer is one which is not associated with or derived from an PC-1 gene 5′ flanking sequence. Examples of a heterologous promoter are the α-fetoprotein, PSA, DF3, tyrosinase, CEA, surfactant protein, and ErbB2 promoters. Examples of a heterologous enhancer are the α-fetoprotein, PSA, DF3, tyrosinase, CEA, surfactant protein, ErbB2, and SV40 enhancers.

An “endogenous” promoter, enhancer, or transcriptional regulatory sequence is native to or derived from adenovirus.

The term “operably linked” relates to the orientation of polynucleotide elements in a functional relationship. A transcriptional regulatory sequence is operably linked to a coding segment if the transcriptional regulatory sequence promotes transcription of the coding sequence. Operably linked means that the DNA sequences being linked are generally contiguous and, where necessary to join two protein coding regions, contiguous and in the same reading frame. However, since enhancers generally function when separated from the promoter by several kilobases and intronic sequences may be of variable length, some polynucleotide elements may be operably linked but not contiguous.

A “host cell” includes an individual cell or cell culture which can be or has been a recipient of any vector of this invention. Host cells include progeny of a single host cell, and the progeny may not necessarily be completed identical (in morphology or in total DNA complement) to the original parent cell due to natural, accidental, or deliberate mutation and/or change. A host cell includes cells transfected or infected in vivo or in vitro with an adenoviral vector of this invention.

A “target cell” is any cell that allows an PC-1 transcriptional regulatory sequence to function. Preferably, a target cell is a mammalian cell which allows an PC-1 transcriptional regulatory sequence to function, such as any cell expressing PC-1, preferably, a mammalian cell endogenously expressing PC-1, more preferably, a human cell, and more preferably, a human cell capable of allowing a PC-1 transcriptional regulatory sequence to function.

As used herein, “neoplastic cells”, “neoplasia”, “tumor”, “tumor cells”, “cancer”, and “cancer cells” refer to cells which exhibit relatively autonomous growth, so that they exhibit an aberrant growth phenotype characterized by a significant loss of control of cell proliferation. Neoplastic cells can be benign or malignant.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples.

An “individual” is a vertebrate, preferably a mammal, more preferably a human. Mammals include, but are not limited to, farm animals, sport animals, and pets.

An “effective amount” is an amount sufficient to effect beneficial or desired clinical results. An effective amount can be administered in one or more administrations. For purposes of this invention, an effective amount of an adenoviral vector is an amount that is sufficient to palliate, ameliorate, stabilize, reverse, slow or delay the progression of the disease state.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, preventing spread (i.e., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment.

“Palliating” a disease means that the extent and/or undesirable clinical manifestations of a disease state are lessened and/or time course of the progression is slowed or lengthened, as compared to not administering adenoviral vectors of the present invention.

Various combinations of transcriptional regulatory sequences can be included in a vector. One or more may be heterologous. Further, one or more may have the tissue-specificity. On or more of the transcriptional regulatory sequences may be inducible. For example, a single transcriptional regulatory sequence could be used to drive replication by more than one gene essential for replication. This is the case, for example, when the gene product of one of the genes drives transcription of the further gene(s). An example is a heterologous promoter linked to a cassette containing an E1a coding sequence (E1a promoter deleted) and the entire E1b gene. In this instance, only one heterologous transcriptional regulatory sequence may be necessary. When genes are individually (separately) controlled, however, more than one transcriptional regulatory sequence can be used if more than one such gene is desired to control replication.

The term “gene essential for replication” refers to a genetic sequence whose transcription is required for the viral vector to replicate in the target cell.

The vectors of the present invention, therefore, also include transcriptional regulatory sequence combinations wherein there is more than one heterologous transcriptional regulatory sequence, but wherein one or more of these is not tissue-specific or tumor-restrictive. For example, one transcriptional regulatory sequence can be a basal level constitutive transcriptional regulatory sequence. For example, a tissue-specific enhancer or promoter can be combined with a basal level constitutive promoter. In another example, a tissue-specific enhancer or promoter can be combined with an inducible promoter.

5 Detailed Description of the Invention

The present invention relates to the isolation, cloning, and characterization of a prostate-specific gene PC-1 (which indicates the first gene identified to be expressed in Prostate and Colon tissues). This gene was found to belong to a gene family with identical sequence at the three prime end of the open reading frame but with distinct identity at the five prime end of the open reading frame. In addition to differences of PC-1 gene profile expression in different tissues, PC-1 gene expression appears to be positively regulated by androgen, whereas the related genes appear to be unregulated by androgen. The PC-1 gene represents a novel prostate-specific gene that could have useful clinical and basic applications.

The present invention additionally relates to methods and compositions for the adenovirus cell therapy. In particular, the compositions of the present invention comprise adenoviral vectors employing a PC-1 transcriptional regulatory sequence to drive viral replication through the regulation of an adenoviral early gene required for viral replication. The methods of the invention involve use of the adenoviral vectors employing a PC-1 transcriptional regulatory sequence which drive viral replication through the regulation of an adenoviral early gene required for viral replication to treat metastatic cancers, including, without limitation, prostate cancer and those diseases involving calcification, including without limitation, benign prostate hyperplasia (BPH).

The preferred vectors of the present invention are adenoviral vectors. In one preferred embodiment, the adenovirus vector is a human adenovirus. There are a number of different types of adenovirus, such as Ad2, Ad5, and Ad40, which may differ to minor or significant degrees. Particularly, Ad5 and Ad40 differ as to their host cell tropism, as well as the nature of the disease induced by the virus.

In another embodiment, the adenovirus vector for use in the compositions and methods of the invention is canine adenovirus type 1 or canine adenovirus type 2. By way of example, and not by way of limitation, examples of canine adenoviruses that may be used are those described in International Patent Application Numbers WO 91/11525 and WO 94/26914, (the entire contents of each of which are incorporated herein by reference).

In another embodiment, the adenovirus vector for use in the compositions and methods of the invention is a bovine adenovirus. By way of example, and not by way of limitation, an example of a bovine adenovirus is that described in International Patent Application Number WO 95/16048 (the entire contents of which are incorporated herein by reference).

In yet another embodiment, the adenovirus vector for use in the compositions and methods of the invention is ovine adenovirus. By way of example, and not by way of limitation, an example of an ovine adenoviral vector suitable for use in the present invention is the ovine adenovirus OAV287 described in U.S. Pat. No. 6,020,172 (the entire contents of which are incorporated herein by reference).

For the purpose of the subject invention, Ad5 will be exemplified. What follows is a brief description of adenovirus-based vectors in general and replication-competent adenovirus vectors in particular.

5.1 Adenovirus-Based Vectors

Adenovirus is a large, non-enveloped virus consisting of a dense protein capsid and a large linear (36 kb) double stranded DNA genome. Adenovirus infects a variety of both dividing and non-dividing cells, gaining entry by receptor-mediated uptake into endosomes, followed by internalization. After uncoating, the adenovirus genome expresses a large number of different gene products that are involved in viral replication, modification of host cell metabolism and packaging of progeny viral particles. Three adenovirus gene products are essential for replication of viral genomes: (1) the terminal binding protein which primes DNA replication, (2) the viral DNA polymerase and (3) the DNA binding protein (reviewed in Tamanoi and Stillman, 1983, Immunol. 109:75-87). In addition, processing of the terminal binding protein by the adenovirus 23 kDa L3 protease is required to permit subsequent rounds of reinfection (Stillman et al., 1981, Cell, 23:497-508) as well as to process adenovirus structural proteins, permitting completion of self-assembly of capsids (Bhatti and Weber, 1979, Virology, 96:478-485).

Packaging of nascent adenovirus particles takes place in the nucleus, requiring both cis-acting DNA elements and trans-acting viral factors, the latter generally construed to be a number of viral structural polypeptides. Packaging of adenoviral DNA sequences into adenovirus capsids requires the viral genomes to possess functional adenovirus encapsidation signals, which are located in the left and right termini of the linear viral genome (Hearing et al., 1987, J. Virol. 61:2555-2558). Additionally, the packaging sequence must reside near the ends of the viral genome to function (Hearing et al., 1987, J. Virol. 61:2555-2558; Grable and Hearing, 1992, J. Virol., 66:723-731). The E1A enhancer, the viral replication origin and the encapsidation signal compose the duplicated inverted terminal repeat (ITR) sequences located at the two ends of adenovirus genomic DNA. The replication origin is defined loosely by a series of conserved nucleotide sequences in the ITR which must be positioned close to the end of the genome to act as a replication-priming element (reviewed in Challberg and Kelly, 1989, Biochem, 58:671-717; Tamanoi and Stillman, 1983, Immunol. 109:75-87). As shown by several groups, the ITRs are sufficient to confer replication to a heterologous DNA in the presence of complementing adenovirus functions. Adenovirus “mini-chromosomes” consisting of the terminal ITRs flanking short linear DNA fragments (in some cases non-viral DNAs) were found to replicate in vivo at low levels in the presence of infecting wild-type adenovirus, or in vitro at low levels in extracts prepared from infected cells (e.g., Hay et al., 1984, J. Mol. Biol. 175:493-510; Tamanoi and Stillman, 1983, Immunol. 109:75-87).

The expression of foreign genes in “replication-defective” adenoviruses (deleted of region E I) has been exploited for a number of years in many labs, and a variety of published reports describe several different approaches often used in constructing these vectors (Vernon et al., 1991, J. Gen. Virol., 72:1243-1251; Wilkinson and Akrigg, 1992, Nuc. Acids Res., 20:2233-2239; Eloit et al., 1990, J. Gen. Virol., 71:2425-2431; Johnson, 1991; Prevec et al., 1990, J. Infect. Dis., 161:27-30; Haj-Ahmad and Graham, 1986, J. Virol., 57:267-274; Lucito and Schneider, 1992, J. Virol., 66:983-991; reviewed in Graham and Prevec, 1992, Butterworth-Heinemann, 363-393). In general, replication-defective viruses are produced by replacing part, or all, of essential region E1 with a heterologous gene of interest, either by direct ligation to viral genomes in vitro, or by homologous recombination within cells in vivo (procedures reviewed in Berkner, 1992, Curr. Topics Micro. Immunol., 158:39-66). These procedures all produce adenovirus vectors that replicate in complementing cell lines such as 293 cells which provide the E1 gene products in trans. Replication competent adenovirus vectors also have been described that have the heterologous gene of interest inserted in place of non-essential region E3 (e.g., Haj-Ahmad and Graham, 1986, J. Virol. 57:267-274), or between the right ITR and region E4 (Saito et al., 1985, J. Virol., 54:711-719). In both, replication defective viruses and replication competent viruses, the heterologous gene of interest is incorporated into viral particles by packaging of the recombinant adenovirus genome.

The E1A gene is expressed immediately after viral infection (0-2 hours) and before any other viral genes. E1A protein acts as a trans-acting positive-acting transcriptional regulatory factor, and is required for the expression of the other early viral genes E1B, E2, E3, E4, and the promoter-proximal major late genes. Despite the nomenclature, the promoter proximal genes driven by the major late promoter are expressed during early times after Ad5 infection. Flint (1982) Biochem. Biophys. Acta 651:175-208; Flint (1986) Advances Virus Research 31:169-228; Grand (1987) Biochem J. 241:25-38. In the absence of a functional E1A gene, viral infection does not proceed, because the gene products necessary for viral DNA replication are not produced. Nevins (1989) Adv. Virus Res. 31:35-81. The transcription start site of Ad5 E1A is at nt 498 and the ATG start site of the E1A protein is at nt 560 in the virus genome.

The E1B protein functions in trans and is necessary for transport of late mRNA from the nucleus to the cytoplasm. Defects in E1B expression result in poor expression of late viral proteins and an inability to shut off host cell protein synthesis. The promoter of E1B has been implicated as the defining element of difference in the host range of Ad40 and Ad5: clinically Ad40 is an enterovirus, whereas Ad5 causes acute conjunctivitis. Bailey et al. (1993) Virology 193:631; Bailey et al. (1994) Virology 202:695-706. E1B proteins are also necessary for the virus to overcome restrictions imposed on viral replication by the host cell cycle and also to reduce the apoptotic effects of E1A. Goodrum et al. (1997) J. Virology 71:548-561. The E1B promoter of Ad5 consists of a single high-affinity recognition site for Sp1 and a TATA box.

The E2 region of adenovirus codes for proteins related to replication of the adenoviral genome, including the 72-kDa DNA-binding protein, the 80-kDa precursor terminal protein and the viral DNA polymerase. The E2 region of Ad5 is transcribed in a rightward orientation from two promoters, termed E2 early and E2 late, mapping at 76.0 and 72.0 map units, respectively. While the E2 late promoter is transiently active during late stages of infection and is independent of the E1A transactivator protein, the E2 early promoter is crucial during the early phases of viral replication.

The E2 early promoter, mapping in Ad5 from 27050-27150, consists of a major and a minor transcription initiation site, the latter accounting for about 5% of the E2 transcripts, two non-canonical TATA boxes, two E2F transcription factor binding sites and an ATF transcription factor binding site. For a detailed review of the E2 promoter architecture see Swaminathan et al., Curr. Topics in Micro. and 1 mm. (1995) 199 part 3:177-194.

The E2 late promoter overlaps with the coding sequences of a gene encoded by the counterstrand and is therefore not amenable for genetic manipulation. However, the E2 early promoter overlaps only for a few base pairs with sequences coding for a 33 kDa protein on the counterstrand. Notably, the SpeI restriction site (Ad5 position 27082) is part of the stop codon for the above mentioned 33 kDa protein and conveniently separates the major E2 early transcription initiation site and TATA-binding protein site from the upstream transcription factor binding sites E2F and ATF. Therefore, insertion of a PC-1 transcriptional regulatory sequence having SpeI ends into the SpeI site in the 1-strand would disrupt the endogenous E2 early promoter of Ad5 and should allow PC-1-restricted expression of E2 transcripts.

The E4 gene produces a number of transcription products. The E4 region codes for two polypeptides which are responsible for stimulating the replication of viral genomic DNA and for stimulating late gene expression. The protein products of open reading frames (ORFs) 3 and 6 can both perform these function by binding the 55-kDa protein from E1B and heterodimers of E2F-1 and DP-1. The ORF 6 protein requires interaction with the E1B 55-kDa protein for activity while the ORF 3 protein does not. In the absence of functional protein from ORF 3 and ORF 6, plaques are produced with an efficiency less than 10-6 that of wild type virus. To further restrict viral replication to cells that allow a PC-1 transcriptional regulatory sequence to function, such as PC-1-producing cells, E4 ORFs 1-3 can be deleted, making viral DNA replication and late gene synthesis dependent on E4 ORF 6 protein. By combining such a vector with sequences in which the E1B region is regulated by a PC-1 transcriptional regulatory sequence, a virus can be obtained in which both the E1B function and E4 function are dependent on a PC-1 transcriptional regulatory sequence driving E1B.

The major late genes relevant to the subject invention are L1, L2, L3, L4, and L5, which encode proteins of the Ad5 virus virion. All of these genes (typically coding for structural proteins) are probably required for adenoviral replication. The late genes are all under the control of the major late promoter (MLP), which is located in Ad5 at about +5986 to about +6048.

In some embodiments, an PC-1 transcriptional regulatory sequence is used with an adenovirus gene that is essential for propagation, so that replication-competence is preferentially achievable in the target cell that allow a PC-1 transcriptional regulatory sequence to function, such as a cell expressing PC-1. Preferably, the gene is an early gene, such as E1A, E1B, E2, or E4. (As noted supra, E3 is not essential for viral replication.) More preferably, the early gene under a PC-1 transcriptional regulatory sequence control is E1 A and/or E1B. More than one early gene can be placed under control of a PC-1 transcriptional regulatory sequence.

In other embodiments, in addition to conferring selective cytotoxic and/or cytolytic activity by virtue of preferential replication competence in cells that allow a PC-1 transcriptional regulatory sequence to function, such as cells expressing PC-1, the adenovirus vectors of this invention can further include a heterologous gene (transgene) under the control of a PC-1 transcriptional regulatory sequence. In this way, various genetic capabilities may be introduced into target cells allowing a PC-1 transcriptional regulatory sequence to function, such as cells expressing PC-1, particularly cancer cells of prostate cancer. For example, in certain instances, it may be desirable to enhance the degree and/or rate of cytotoxic activity, due to, for example, the relatively refractory nature or particular aggressiveness of the PC-1-producing target cell. This could be accomplished by coupling the cell-specific replicative cytotoxic activity with cell-specific expression of, for example, HSV-tk and/or cytosine deaminase (cd), which renders cells capable of metabolizing 5-fluorocytosine (5-FC) to the chemotherapeutic agent 5-fluorouracil (5-FU). Using these types of heterologous genes or transgenes may also confer a bystander effect.

Other desirable transgenes that may be introduced via an adenovirus vector(s) include genes encoding cytotoxic proteins, such as the A chains of diphtheria toxin, ricin or abrin [Palmiter et al. (1987) Cell 50:435; Maxwell et al. (1987) Mol. Cell. Biol. 7:1576;

Behringer et al. (1988) Genes Dev. 2:453; Messing et al. (1992) Neuron 8:507; Piatak et al. (1988) J. Biol. Chem. 263:4937; Lamb et al. (1985) Eur. J. Biochem. 148:265; Frankel et al. (1989) Mo. Cell. Biol. 9:415], genes encoding a factor capable of initiating apoptosis, sequences encoding antisense transcripts or ribozymes, which among other capabilities may be directed to mRNAs encoding proteins essential for proliferation, such as structural proteins, or transcription factors; viral or other pathogenic proteins, where the pathogen proliferates intracellularly, genes that encode an engineered cytoplasmic variant of a nuclease (e.g. RNase A) or protease (e.g. awsin, papain, proteinase K, carboxypeptidase, etc.), or encode the Fas gene, and the like. Other genes of interest include cytokins, antigens, transmembrane proteins, and the like, such as IL-1, -2, -6, -12, GM-CSF, G-CSF, M-CSF, IFN-.alpha., -.beta., -.gamma., TNF-.alpha., -.beta., NGF, and the like. The positive effector genes could be used in an early phase, followed by cytotoxic activity due to replication. In alternative embodiments, adenovirus vectors are provided with any of the other genes essential for replication, such as, for example, but not limited to, E2 or E4, under the control of a heterologous transcriptional regulatory sequence.

With respect to the packaging capacity of the adenovirus vectors of the present invention, if no adenovirus sequences have been deleted, an adenoviral vector can be packaged with extra sequences totaling up to about 5% of the genome size, or approximately 1.8 kb. If non-essential sequences are removed from the adenovirus genome, then an additional 4.6 kb of insert can be accommodated (i.e., a total of about 1.8 kb plus 4.6 kb, which is about 6.4 kb). Examples of non-essential adenoviral sequences that can be deleted are E3 and E4 (as long as the E4 ORF6 is maintained).

Any of the adenoviral vectors described herein can be used in a variety of forms, including, but not limited to, naked polynucleotide (usually DNA) constructs. Adenoviral vectors can, alternatively, comprise polynucleotide constructs that are complexed with agents to facilitate entry into cells, such as cationic liposomes or other compounds such as polylysine; packaged into infectious adenovirus particles (which may render the adenoviral vector(s) more immunogenic); complexed with agents to enhance or dampen an immune response; or complexed with agents that facilitate in vivo transfection, such as DOTMA, DOTAP.TM., and polyamines.

The adenoviral vectors may be delivered to the target cell in a variety of ways, including, but not limited to, liposomes, general transfection methods that are well known in the art, such as calcium phosphate precipitation, electroporation, direct injection, and intravenous infusion. The means of delivery will depend in large part on the particular adenoviral vector (including its form) as well as the type and location of the target cells (i.e., whether the cells are in vitro or in vivo).

If used in packaged adenoviruses, adenovirus vectors may be administered in an appropriate physiologically acceptable carrier at a dose of about 10¹⁴ PFU to about 10¹⁴ PFU. The multiplicity of infection will generally be in the range of about 0.001 PFU to 100 PFU. If administered as a polynucleotide construct (i.e., not packaged as a virus) about 0.01 μg to 1000 μg of an adenoviral vector can be administered. The adenoviral vector(s) may be administered one or more times, depending upon the intended use and the immune response potential of the host or may be administered as multiple simultaneous injections. If an immune response is undesirable, the immune response may be diminished by employing a variety of immunosuppressants, so as to permit repetitive administration, without a strong immune response.

The present invention also includes compositions, including pharmaceutical compositions, containing the adenoviral vectors described herein. Such compositions are useful for administration in vivo, for example, when measuring the degree of transduction and/or effectiveness of cell killing in an individual. Preferably, these compositions further comprise a pharmaceutically acceptable excipient. These compositions, which can comprise an effective amount of an adenoviral vector of this invention in a pharmaceutically acceptable excipient, are suitable for systemic administration to individuals in unit dosage forms, sterile parenteral solutions or suspension, sterile non-parenteral solutions or oral solutions or suspensions, oil in water or water in oil emulsions and the like. Formulations for parenteral and nonparenteral drug delivery are known in the art and are set forth in Remington's Pharmaceutical Sciences, 18th Edition, Mack Publishing (1990). Compositions also include lyophilized and/or reconstituted forms of the adenoviral vectors (including those packaged as a virus, such as adenovirus) of the invention.

The present invention also encompasses kits containing an adenoviral vector of this invention. These kits can be used for diagnostic and/or monitoring purposes, preferably monitoring. Procedures using these kits can be performed by clinical laboratories, experimental laboratories, medical practitioners, or private individuals. Kits embodied by this invention allow for the detection of the presence of cells that allow a PC-1 transcriptional regulatory sequence to function, such as PC-1-producing cells in a suitable biological sample, such as biopsy specimens.

The kits of the invention comprise an adenoviral vector described herein in suitable packaging. The kit may optionally provide additional components that are useful in the procedure, including, but not limited to, buffers, developing reagents, labels, reacting surfaces, means for detection, control samples, instructions, and interpretive information.

5.2 Methods Using the Adenovirus Vectors of the Invention

The subject vectors can be used for a wide variety of purposes, which will vary with the desired or intended result. Accordingly, the present invention includes methods using the adenoviral vectors described above.

In one embodiment, methods are provided for conferring selective cytotoxicity in cells which allow a PC-1 transcriptional regulatory sequence to function, such as cells expressing PC-1 comprising contacting the cells with an adenovirus vector described herein. Cytotoxicity can be measured using standard assays in the art, such as dye exclusion, H-thymidine incorporation, and/or lysis.

In another embodiment, methods are provided for propagating an adenovirus specific for cells that allow a PC-1 transcriptional regulatory sequence to function, such as those cells expressing PC-1. These methods entail infecting cells with an adenovirus vector whereby said adenovirus is propagated.

Another embodiment provides methods of killing cells that allow a PC-1 transcriptional regulatory sequence to function, such as cells expressing PC-1 in a mixture of cells, comprising infecting a mixture of cells with an adenovirus vector of the present invention. The mixture of cells is generally a mixture of normal cells and cancerous cells producing PC-1, and can be an in vivo mixture or in vitro mixture.

The invention also includes methods for detecting cells which allow a PC-1 transcriptional regulatory sequence to function, such as cells expressing PC-1 in a biological sample. These methods are particularly useful for monitoring the clinical and/or physiological condition of an individual (i.e., mammal), whether in an experimental or clinical setting. In one method, cells of a biological sample are contacted with an adenovirus vector, and replication of the adenoviral vector is detected. Alternatively, the sample can be contacted with an adenovirus in which a reporter gene is under control of a PC-1 transcriptional regulatory sequence. Expression of the reporter gene indicates the presence of cells that allow the PC-1 transcriptional regulatory sequence to function, such as PC-1-producing cells. Non-limiting examples of reporter genes for use in the methods of the invention include luciferase, and beta-galactosidase.

The transcriptional activation or increase in transcription that is observed in such PC-1-producing cells, is that transcription which will be increased above basal levels in the target cell (i.e. cells that allow a PC-1 transcriptional regulatory sequence to function, such as, for example, without limitation, prostate cancer cells, prostate stromal cells, breast cancer cells, renal cells, bladder cells, and cells of the endometrium by at least about 20-fold, more preferably at least about 50-fold, more preferably at least about 100-fold, even more preferably at least about 200-fold, even more preferably at least about 400- to about 500-fold, even more preferably, at least about 1000-fold. In some cases the increase in transcription may be about 2-fold, about 5-fold, or about 10-fold over basal levels in the target cell.

Alternatively, an adenovirus can be constructed in which a gene conditionally required for cell survival is placed under control of a PC-1 transcriptional regulatory sequence. This gene may encode, for example, antibiotic resistance. The adenovirus is introduced into the biological sample, and at a later time interval the sample is treated with an antibiotic. The presence of surviving cells expressing antibiotic resistance indicates the presence of cells that allow a PC-1 transcriptional regulatory sequence to function. A suitable biological sample is one in which PC-1-producing cells may be or are suspected to be present. Generally, in mammals, a suitable clinical sample is one in which cancerous cells producing PC-1, such as prostate cancer cells, prostate stromal cells, breast cancer cells, renal cells, bladder cells, and cells of the endometrium, are suspected to be present. Such cells can be obtained, for example, by needle biopsy or by any other suitable surgical procedure. Cells to be contacted may be treated to promote assay conditions such as selective enrichment and/or solubilization. In these methods, PC-1-producing cells can be detected using in vitro assays that detect proliferation, which are standard in the art. Examples of such standard assays include, but are not limited to, burst assays (which measure virus yields) and plaque assays (which measure infectious particles per cell). Also, propagation can be detected by measuring specific adenoviral DNA replication, which are also standard assays.

The invention also provides methods of modifying the genotype of a target cell, comprising contacting the target cell with an adenovirus vector described herein, wherein the adenoviral vector enters the cell.

The invention further provides methods of suppressing tumor cell growth, preferably a tumor cell that expresses PC-1, comprising contacting tumor cells and non tumor cells with an adenoviral vector of the invention such that the adenoviral vector enters the tumor cell and exhibits selective cytotoxicity for the tumor cell. Tumor cell growth can be assessed by any means known in the art, including, but not limited to, measuring tumor size, determining whether tumor cells are proliferating using a ³H-thymidine incorporation assay, or counting tumor cells. “Suppressing” tumor cell growth means any or all of the following states: slowing, delaying, and stopping tumor growth, as well as tumor shrinkage. “Suppressing” tumor growth indicates a growth state that is curtailed when compared to growth without contact with, i.e., transfection by, an adenoviral vector described herein.

The invention also provides methods of lowering the levels of a tumor cell marker in an individual, comprising administering to the individual an adenoviral vector of the present invention, wherein the adenoviral vector is selectively cytotoxic toward cells producing the tumor cell marker. Tumor cell markers include, but are not limited to, PSA, hK2, and carcinoembryonic antigen. Methods of measuring the levels of a tumor cell marker are known to those of ordinary skill in the art and include, but are not limited, to, immunological assays, such as enzyme-linked immunosorbent assay (ELISA), using antibodies specific for the tumor cell marker. In general, a biological sample is obtained from the individual to be tested, and a suitable assay, such as an ELISA, is performed on the biological sample.

The invention also provides methods of treatment, in which an effective amount of an adenoviral vector(s) described herein is administered to an individual. Treatment using an adenoviral vector(s) is indicated in individuals with metastatic cancers, including, without limitation, prostate cancer, brain cancer, ovarian cancer, thyroid cancer, tumors, renal carcinoma, bladder cancer, lung cancer, or breast cancer. Also indicated are individuals who are considered to be at risk for developing metastatic cancers, including, without limitation, prostate cancer, brain cancer, ovarian cancer, thyroid cancer, tumors, renal carcinoma, bladder cancer, lung cancer, and breast cancer-associated diseases, such as those who have had disease which has been resected and those who have had a family history of metastatic cancers, including, without limitation, prostate cancer, brain cancer, ovarian cancer, thyroid cancer, tumors, lung cancer, renal carcinoma, bladder cancer and breast cancer-associated diseases. Determination of suitability of administering adenoviral vector(s) of the invention will depend, inter alia, on assessable clinical parameters such as serological indications and histological examination of tissue biopsies. Generally, a pharmaceutical composition comprising an adenoviral vector(s) in a pharmaceutically acceptable excipient is administered. Pharmaceutical compositions are described above.

The amount of adenoviral vector(s) to be administered will depend on several factors, such as route of administration, the condition of the individual, the degree of aggressiveness of the disease, the particular PC-1 transcriptional regulatory sequence employed, and the particular vector construct (i.e., which adenovirus gene(s) is under PC-1 transcriptional regulatory sequence control).

If administered as a packaged adenovirus, from about 10⁴ PFU to about 10¹⁴ PFU, preferably from about 10⁴ PFU to about 10¹² PFU, more preferably from about 10⁴ PFU to about 10¹⁰ PFU. If administered as a polynucleotide construct (i.e., not packaged as a virus), about 0.01 μg to about 100 μg can be administered, preferably 0.1 μg to about 500 μg, more preferably about 0.5 μg to about 200 μg. More than one adenoviral vector can be administered, either simultaneously or sequentially, Administrations are typically given periodically, while monitoring any response. Administration can be given, for example, intratumorally, intravenously or intraperitoneally.

The adenoviral vectors of the invention can be used alone or in conjunction with other active agents, such as chemotherapeutics, that promote the desired objective.

In accordance with the present invention, the agent which is capable of providing for the inhibition, prevention, or destruction of the growth of the target tissue or tumor cells upon expression of such agent can thus also be a negative selective marker which is provided as a heterologous gene or transgene; i.e., a material which in combination with a chemotherapeutic or interaction agent inhibits, prevents or destroys the growth of the target cells.

Thus, upon introduction to the cells of the negative selective marker, an interaction agent is administered to the host. The interaction agent interacts with the negative selective marker to prevent, inhibit, or destroy the growth of the target cells.

Negative selective markers which may be used in the methods of the present invention include, but are not limited to, thymidine kinase and cytosine deaminase. In one embodiment, the negative selective marker is a viral thymidine kinase selected from the group consisting of Herpes simplex virus thymidine kinase, cytomegalovirus thymidine kinase, and varicella-zoster virus thymidine kinase. When viral thymidine kinases are employed, the interaction or chemotherapeutic agent preferably is a nucleoside analogue, for example, one selected from the group consisting of ganciclovir, acyclovir, and 1-2-deoxy-2-fluoro-.beta.-D-arabinofuranosil-5-iodouracil (FIAU). Such interaction agents are utilized efficiently by the viral thymidine kinases as substrates, and such interaction agents thus are incorporated lethally into the DNA of the tumor cells expressing the viral thymidine kinases, thereby resulting in the death of the target cells.

When cytosine deaminase is the negative selective marker, a preferred interaction agent is 5-fluorocytosine. Cytosine deaminase converts 5-fluorocytosine to 5-fluorouracil, which is highly cytotoxic. Thus, the target cells which express the cytosine deaminase gene convert the 5-fluorocytosine to 5-fluorouracil and are killed.

The interaction agent is administered in an amount effective to inhibit, prevent, or destroy the growth of the target cells. For example, the interaction agent is administered in an amount based on body weight and on overall toxicity to a patient. The interaction agent preferably is administered systemically, such as, for example, by intravenous administration, by parenteral administration, by intraperitoneal administration, or by intramuscular administration.

When the vectors of the present invention induce a negative selective marker and are administered to a tissue or tumor in vivo, a “bystander effect” may result, i.e., cells which were not originally transduced with the nucleic acid sequence encoding the negative selective marker may be killed upon administration of the interaction agent. Although the scope of the present invention is not intended to be limited by any theoretical reasoning, the transduced cells may be producing a diffusible form of the negative selective marker that either acts extracellularly upon the interaction agent, or is taken up by adjacent, non-target cells, which then become susceptible to the action of the interaction agent. It also is possible that one or both of the negative selective marker and the interaction agent are communicated between target cells.

In one embodiment, the agent which provides for the inhibition, prevention, or destruction of the growth of the tumor cells is a cytokine. In one embodiment, the cytokine is an interleukin. Other cytokines which may be employed include interferons and colony-stimulating factors, such as GM-CSF. Interleukins include, but are not limited to, interleukin-1, interleukin-1β, and interleukins-2-15. In one embodiment, the interleukin is interleukin-2.

In a preferred embodiment of the invention, the target tissue is that of metastatic cancers, including, without limitation, prostate cancer, brain cancer, ovarian cancer, thyroid cancer, tumors, lung cancer, renal carcinoma, bladder cancer, breast cancer and those diseases involving calcification, including, without limitation, benign prostate hyperplasia (BPH). The virus is distributed throughout the tissue or tumor mass. In another preferred embodiment the target tissue comprises cells which allow a PC-1 transcriptional regulatory sequence to function, such as for example, but not limited to, prostate cancer cells, prostate stromal cells, breast cancer cells, renal cells, bladder cells, and cells of the endometrium. The virus is distributed throughout the tissue or tumor mass.

5.3 Additional Embodiments of the Invention

In another embodiment, the invention additionally comprises using the adenoviral compositions and methods of the present invention in combination with a gene therapy method for treating prostate cancer. Tissue specific and tumor-restrictive promoters such as the PC-1 transcriptional regulatory sequence promoter sequence comprising nucleotides 1-322 of SEQ ID NO:1 or any other tissue specific promoter described supra are used to drive tissue-specific and tumor-restrictive expression of therapeutic molecules and introduced in the cells of the cancer. The method comprises introducing an adenoviral vector constructed with an essential gene under the control of a tissue specific promoter such as the PC-1 transcriptional regulatory sequence, wherein the adenoviral vector additionally contains another tissue-specific promoter operatively associated with a nucleic acid encoding a therapeutic molecule, into cells of the cancer, including, for example, without limitation, such cancers as prostate cancer, brain cancer, ovarian cancer, thyroid cancer, tumors, renal carcinoma, bladder cancer, lung cancer, and breast cancer.

Specifically provided are expression vectors comprising the PC-1 transcriptional regulatory sequence, and transcriptionally active fragments thereof, operably associated to a heterologous reporter gene, e.g., LacZ, and host cells and transgenic animals containing such vectors. The invention also provides methods for using such vectors, cells and animals for screening candidate molecules for agonists and antagonists of prostate-related disorders. Methods for using molecules and compounds identified by the screening assays for therapeutic treatments also are provided.

For example, and not by way of limitation, a composition comprising a reporter gene is operatively linked to a PC-1 transcriptional regulatory sequence. The tissue specific promoter such as PC-1 promoter driven reporter gene is expressed as a transgene in animals. The transgenic animal, and cells derived from the prostate of such a transgenic animal, can be used to screen compounds for candidates useful for modulating prostate-related disorders and diseases involving calcification. Without being bound by any particular theory, such compounds are likely to interfere with the function of trans-acting factors, such as transcription factors, cis-acting elements, such as promoters and enhancers, as well as any class of post-transcriptional, translational or post-translational compounds involved in prostate-related disorders and diseases involving calcification. As such, they are powerful candidates for treatment of such cancers and disorders.

In one embodiment, the invention provides methods for high throughput screening of compounds that modulate specific expression of genes within cells of prostate cancer, brain cancer, ovarian cancer, thyroid cancer, tumors, lung cancer, renal carcinoma, bladder cancer, and breast cancer. In this aspect of the invention, cells from the prostate cancer, brain cancer, ovarian cancer, thyroid cancer, tumors, lung cancer, renal carcinoma, bladder cancer, and breast cancer. The expression of the reporter gene is used to monitor PC-1-specific gene activity. In a specific embodiment, LacZ is the reporter gene. In another specific embodiment, luciferase is the reporter gene. Compounds identified by this method can be tested further for their effect on prostate cancer, brain cancer, ovarian cancer, thyroid cancer, tumors, lung cancer, renal carcinoma, bladder cancer, and breast cancer.

In another embodiment, the transgenic animal models of the invention can be used for in vivo screening to test the mechanism of action of candidate drugs for their effect on prostate cancer, brain cancer, ovarian cancer, thyroid cancer, tumors, lung cancer, renal carcinoma, bladder cancer, and breast cancer.

Specifically, the effects of the drugs on prostate cancer, brain cancer, ovarian cancer, thyroid cancer, tumors, lung cancer, renal carcinoma, bladder cancer, and breast cancer.

5.4 Polynucleotides and Nucleic Acids of the Invention

The present invention encompasses polynucleotide sequences comprising 5′ regulatory regions, and transcriptionally active fragments thereof, of the PC-1 transcriptional regulatory sequence. In particular, the present invention provides a polynucleotide comprising the PC-1 promoter sequence comprising nucleotides 1-322 of SEQ ID NO:1, and transcriptionally active fragments thereof.

The invention further provides probes, primers and fragments of a tissue specific promoter such as the PC-1 promoter sequence depicted in SEQ ID NO:1. In one embodiment, purified nucleic acids consisting of at least 8 nucleotides (i.e., a hybridizable portion) of a tissue specific promoter such as a PC-1 regulatory sequence are provided; in other embodiments, the nucleic acids consist of at least 20 (contiguous) nucleotides, 25 nucleotides, 50 nucleotides, 100 nucleotides, 200 nucleotides or 500 nucleotides of a tissue specific promoter such as the PC-1 promoter sequence promoter sequence comprising nucleotides 1-322 of SEQ ID NO:1. Methods which are well known to those skilled in the art can be used to construct these sequences, either in isolated form or contained in expression vectors. These methods include, for example, in vitro recombinant DNA techniques, synthetic techniques and in vivo genetic recombination. See, e.g., the techniques described in Sambrook et al., 1989, supra, and Ausabel et al., 1989, supra; also see the techniques described in “Oligonucleotide Synthesis”, 1984, Gait M. J. ed., IRL Press, Oxford, which is incorporated herein by reference in its entirety.

In another embodiment, the nucleic acids are smaller than 20, 25, 35, 200 or 500 nucleotides in length. Nucleic acids can be single or double stranded. The invention also encompasses nucleic acids hybridizable to or complementary to the foregoing sequences. In specific aspects, nucleic acids are provided which comprise a sequence complementary to at least 10, 20, 25, 50, 100, 200, 500 nucleotides or the entire regulatory region of a tissue specific promoter such as the PC-1 promoter sequence comprising nucleotides 1-322 of SEQ ID NO:1.

The probes, primers and fragments of the tissue specific promoter such as the promoter sequence comprising nucleotides 1-322 of SEQ ID NO:1 provided by the present invention can be used by the research community for various purposes. They can be used as molecular weight markers on Southern gels; as chromosome markers or tags (when appropriately labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; and as a probe to “subtract-out” known sequences in the process of discovering other novel polynucleotides. Methods for performing the uses listed above are well known to those skilled in the art. References disclosing such methods include, without limitation, “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

The nucleotide sequences of the invention also include nucleotide sequences that have at least 65%, 70%, 75%, 80%, 85%, 90%, 95%, 98%, 99% or more nucleotide sequence identity to the PC-1 gene sequence depicted in SEQ ID NO:1.

The nucleotide sequences of the invention also include nucleotide sequences that have at least 65%, 70%, 75%, 80%, 85%, 90%, 95%, 98%, 99% or more nucleotide sequence identity to the PC-1 promoter sequence comprising nucleotides 1-322 of SEQ ID NO:1, and/or transcriptionally active fragments thereof.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared.

When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of positions×100). In one embodiment, the two sequences are the same length.

The determination of percent identity between two sequences also can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12 and a gap penalty of 4 can be used. In an alternate embodiment, alignments can be obtained using the NA-MULTIPLE-ALIGNMENT 1.0 program, using a Gap Weight of 5 and a Gap Length Weight of 1.

The percent identity between two sequences can be determined using techniques similar to those described above, with or without allowing gaps. In calculating percent identity, typically only exact matches are counted.

The invention also encompasses:

(a) DNA vectors that contain any of the foregoing tissue specific promoter such as the PC-1 promoter sequence comprising nucleotides 1-322 of SEQ ID NO:1 and/or their complements (i.e., antisense);

(b) DNA expression vectors that contain any of the foregoing tissue specific promoter such as the PC-1 promoter sequence comprising nucleotides 1-322 of SEQ ID NO:1 operatively associated with a heterologous gene, such as a reporter gene;

(c) genetically engineered host cells that contain any of the foregoing tissue specific promoter such as the PC-1 promoter sequences depicted in SEQ ID NO:1 operatively associated with a heterologous gene such that the tissue specific promoter such as PC-1 promoter element directs the expression of the heterologous gene in the host cell.

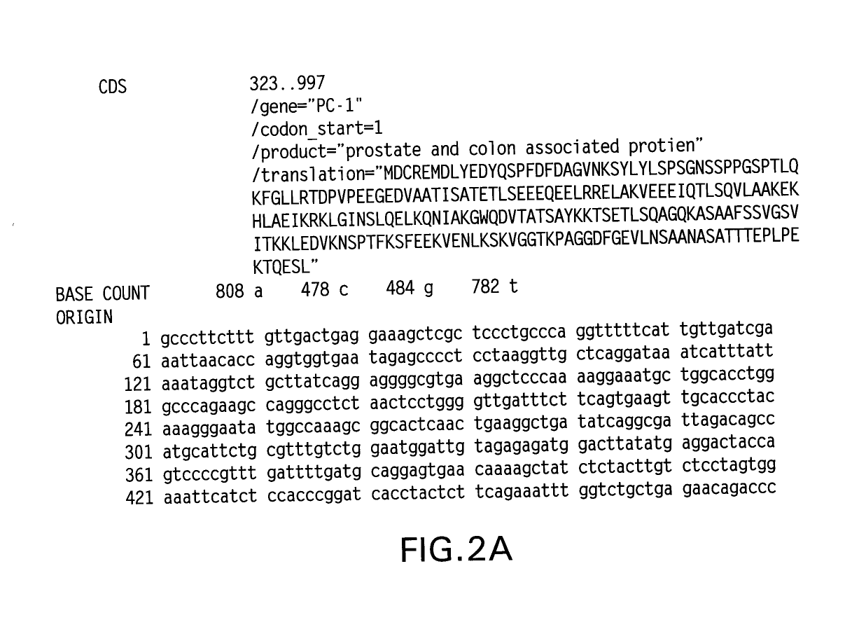

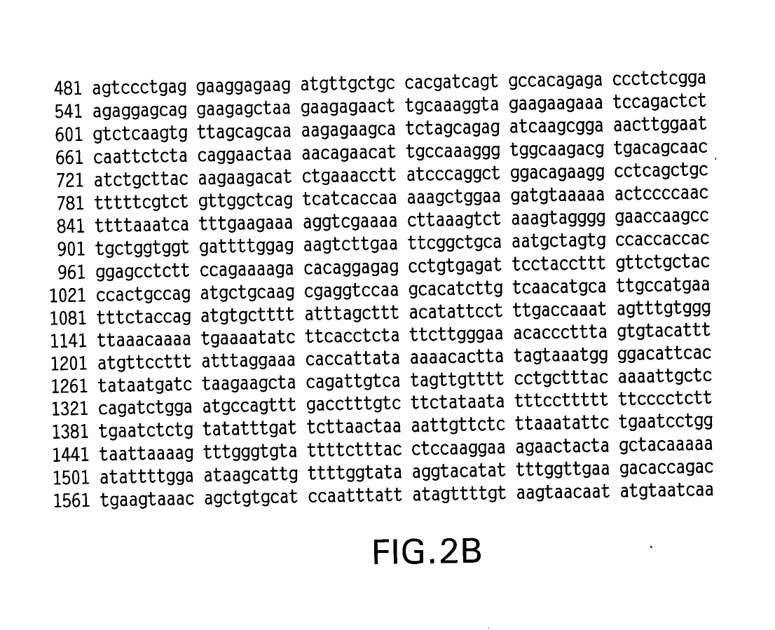

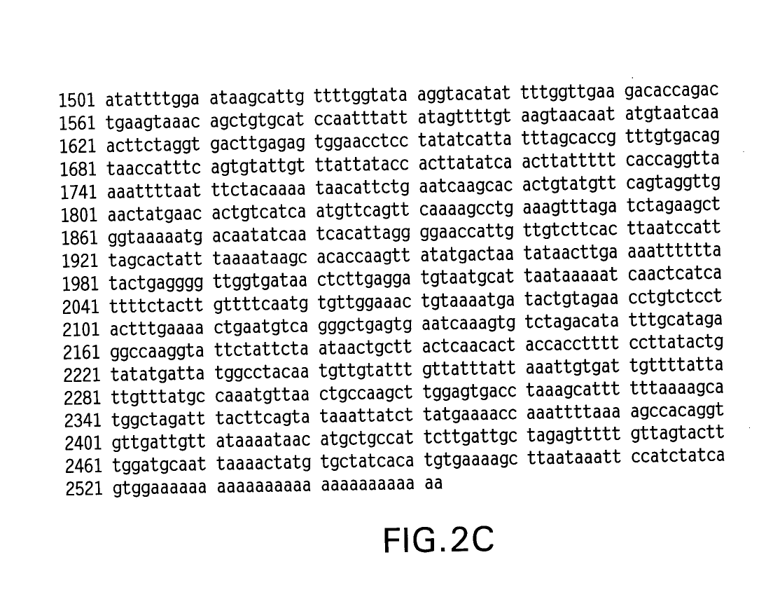

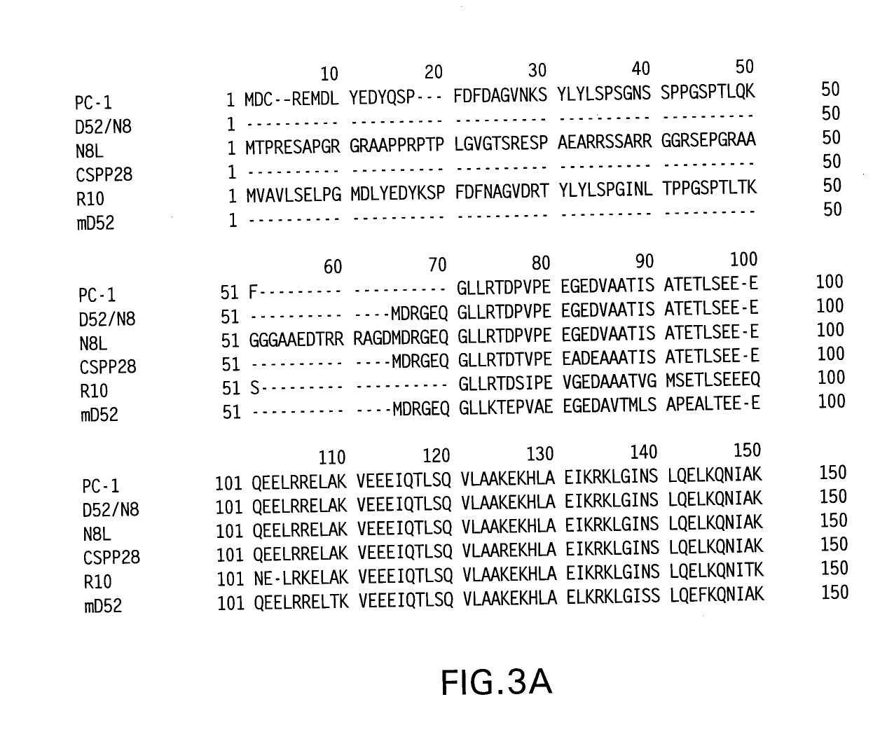

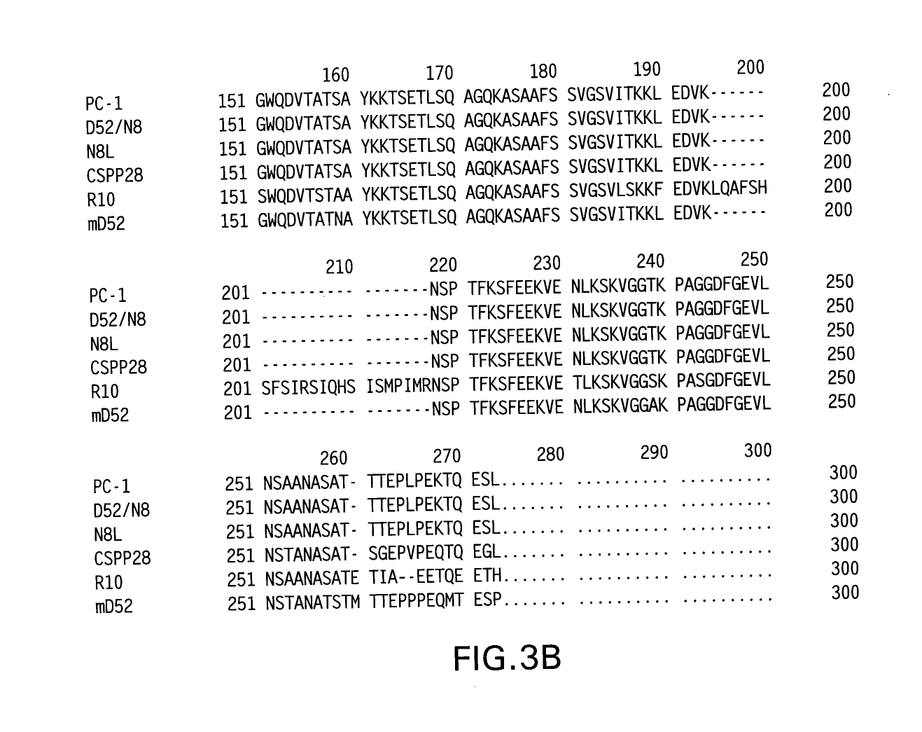

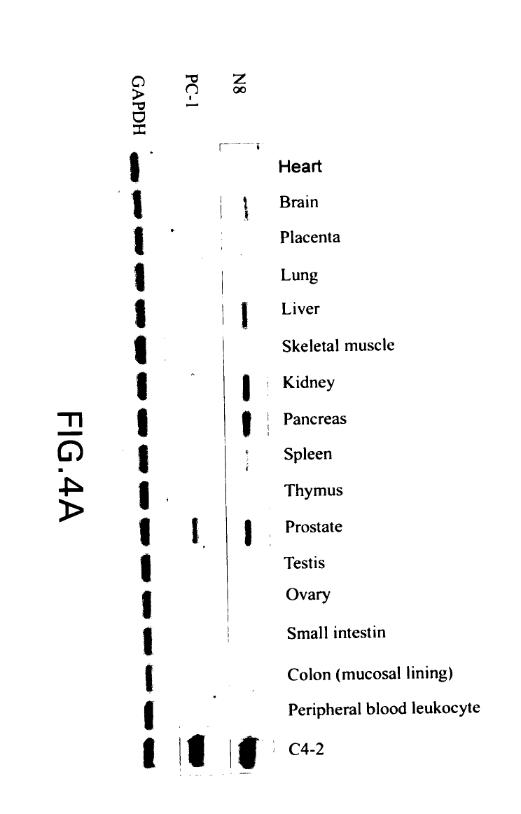

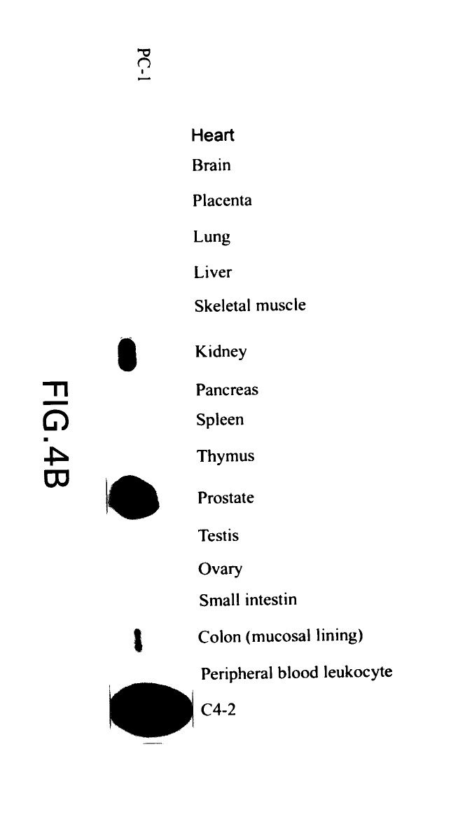

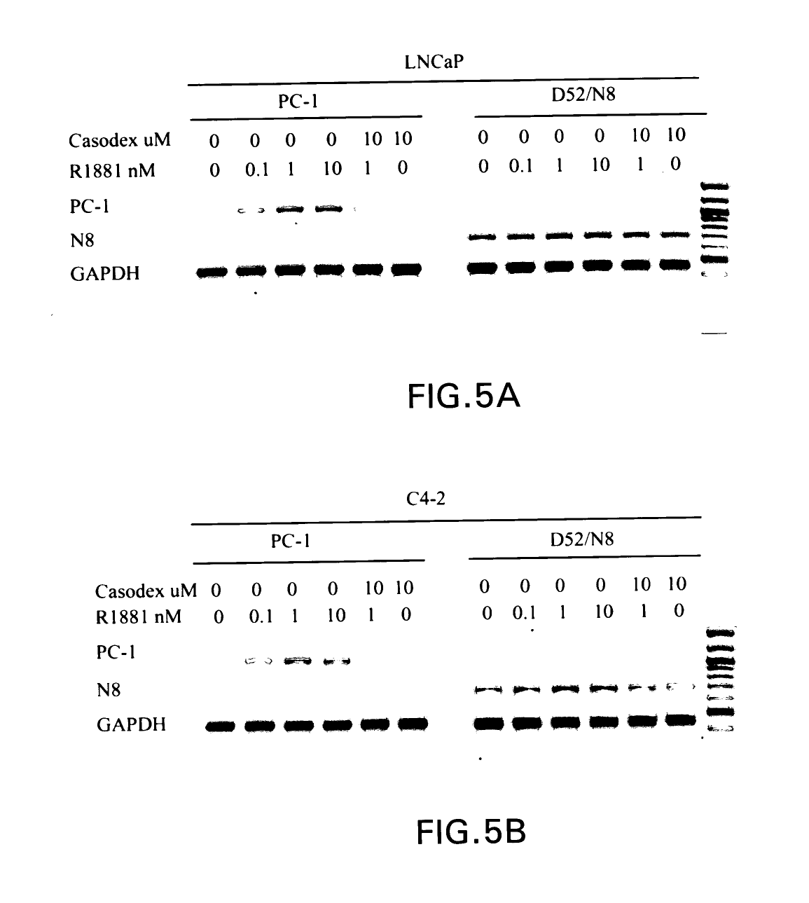

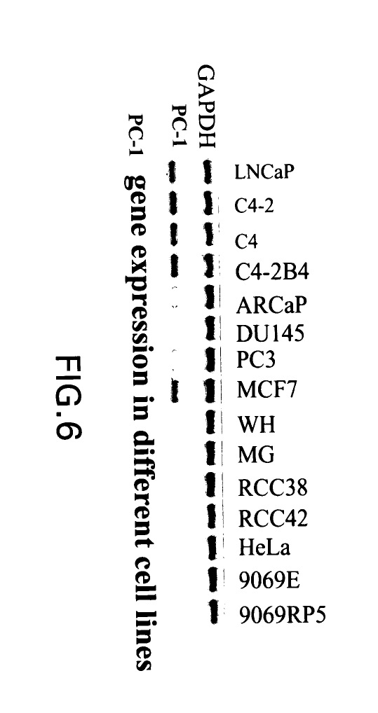

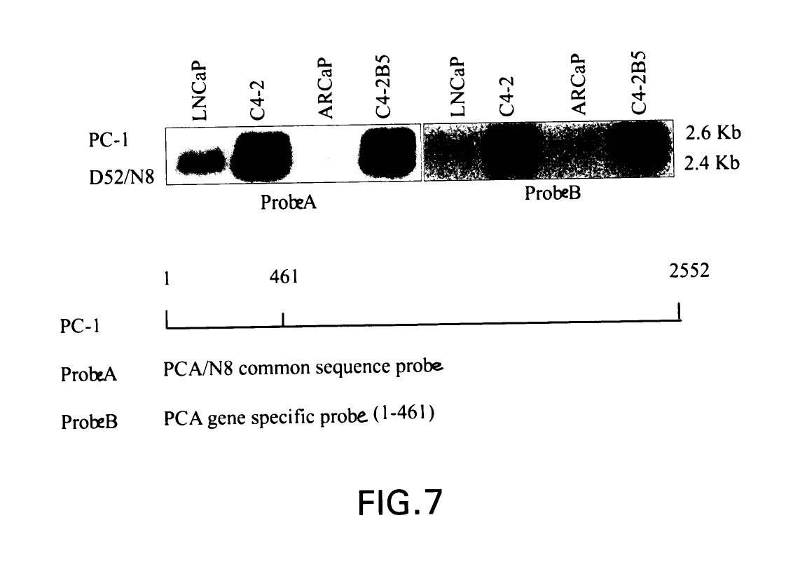

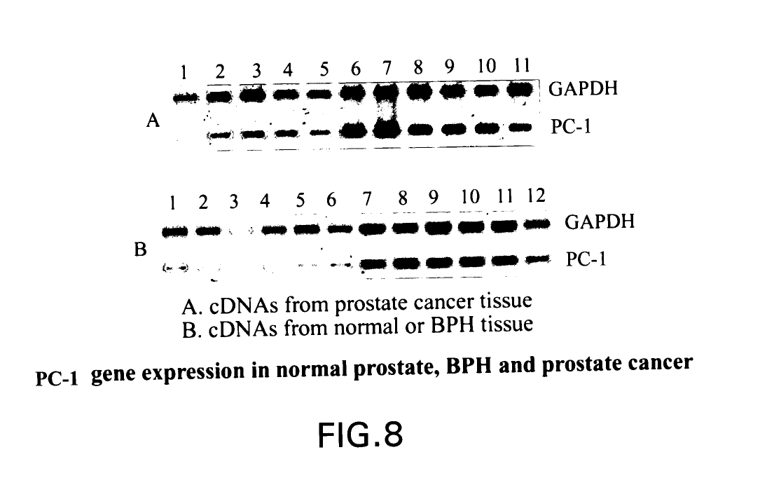

(d) a nucleic acid molecule comprising a PC-1 nucleic acid sequence (e.g., the nucleic acid sequences depicted in FIG. 2, or a fragment thereof);

(e) a nucleic acid molecule that encodes a PC-1 gene product, such as a nucleic acid molecule that encodes a polypeptide comprising the amino acid sequence shown in FIG. 2;

(f) a nucleic acid molecule comprising a PC-1 nucleotide sequence that encodes a mutant of a PC-1 gene product in which all or a part of a domain is deleted or altered, as well as fragments thereof;

(g) nucleic acid molecules that encode fusion proteins comprising a PC-1 gene product (e.g., amino acid sequences shown in FIG. 2), or a fragment thereof, fused to a heterologous polypeptide;

(h) nucleic acid molecules within a PC-1 sequence described in b), above (e.g., primers), or within chromosomal nucleotide sequences flanking the PC-1 gene, and which can be utilized as part of the methods of the invention for identifying and diagnosing individuals at risk for or exhibiting a PC-1-related disorder, such as prostate cancer.

Also encompassed within the scope of the invention are various transcriptionally active fragments of this regulatory region. A “transcriptionally active” or “transcriptionally functional” fragment of a tissue specific promoter such as the PC-1 regulatory sequence comprising nucleotides 1-322 of SEQ ID NO:1 according to the present invention refers to a polynucleotide comprising a fragment of said polynucleotide which is functional as a regulatory region for expressing a recombinant polypeptide or a recombinant polynucleotide in a recombinant cell host. For the purpose of the invention, a nucleic acid or polynucleotide is “transcriptionally active” as a regulatory region for expressing a recombinant polypeptide or a recombinant polynucleotide if said regulatory polynucleotide contains nucleotide sequences which contain transcriptional information, and such sequences are operably associated to nucleotide sequences which encode the desired polypeptide or the desired polynucleotide.

In particular, the transcriptionally active fragments of the tissue specific promoter such as the PC-1 regulatory sequence comprising nucleotides 1-322 of SEQ ID NO:1 of the present invention encompass those fragments that are of sufficient length to promote transcription of a heterologous gene, such as a reporter gene, when operatively linked to the tissue specific promoter such as the PC-1 regulatory sequence comprising nucleotides 1-322 of SEQ ID NO:1 and transfected into a prostate cell line. Typically, the regulatory region is placed immediately 5′ to, and is operatively associated with the coding sequence. As used herein, the term “operatively associated” refers to the placement of the regulatory sequence immediately 5′ (upstream) of the reporter gene, such that trans-acting factors required for initiation of transcription, such as transcription factors, polymerase subunits and accessory proteins, can assemble at this region to allow RNA polymerase dependent transcription initiation of the reporter gene.

In one embodiment, the polynucleotide sequence chosen to serve as the tissue-specific transcriptional regulatory sequence may further comprise other nucleotide sequences in addition to those of the PC-1 regulatory sequence comprising nucleotides 1-322 of SEQ ID NO:1. Such tissue-specific transcriptional regulatory sequence may include, for example, without limitation, the α-fetoprotein, PSA, DF3, tyrosinase, CEA, surfactant protein, and ErbB2 promoters. In particularly preferred embodiments, the additional tissue-specific transcriptional regulatory sequence may include for example, without limitation, the PSA promoter, the prostate specific enhancer (PSE), superPSE promoter, the modified artificial α-fetoprotein promoter sequence described in U.S. Pat. No. 5,998,205, the entire contents of which are incorporated by reference), the modified artificial α-fetoprotein promoter sequence described in Hallebbeck et al. (Hallenbeck et al. 1999 Human Gene Therapy 10: 1721-1733, the entire contents of which are incorporated by reference) and the modified artificial α-fetoprotein promoter described in Nakabayashi et al. (Mol. Cell. Biol. 1991 11(12):5885-93, the entire contents of which are incorporated by reference).

In yet another embodiment, multiple copies of the PC-1 regulatory sequence comprising nucleotides 1-322 of SEQ ID NO:1, or a fragment thereof, may be linked to each other. For example, the PC-1 regulatory sequence comprising nucleotides 1-322 of SEQ ID NO:1, or a fragment thereof, may be linked to another copy of the promoter sequence, or another fragment thereof, in a head to tail, head to head, or tail to tail orientation. Thus, in another embodiment, by way of example, and not by way of limitation, a prostate cell-specific enhancer may be operatively linked to the tissue specific promoter such as the PC-1 regulatory sequence comprising nucleotides 1-322 of SEQ ID NO:1, or fragment thereof, and used to enhance transcription from the construct containing the tissue specific PC-1 regulatory sequence comprising nucleotides 1-322 of SEQ ID NO:1.

Also encompassed within the scope of the invention are modifications of the PC-1 regulatory sequence comprising nucleotides 1-322 of SEQ ID NO:1 without substantially affecting its transcriptional activities. Such modifications include additions, deletions and substitutions. In addition, any nucleotide sequence that selectively hybridizes to the complement of the PC-1 regulatory sequence comprising nucleotides 1-322 of SEQ ID NO:1 under stringent conditions, and is capable of activating the expression of a gene essential for replication of the adenovirus is encompassed by the invention. Exemplary moderately stringent hybridization conditions are as follows: prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/mQ denatured salmon sperm DNA. Filters are hybridized for 48 hours at 65° C. in prehybridization mixture containing 100 μg/mQ denatured salmon sperm DNA and 5-20×10⁶ cpm of ³²P-labeled probe. Washing of filters is done at 37° C. for 1 hour in a solution containing 2×SSC, 0.01% PVP, 0.01% Ficoll, and 0.01% BSA. This is followed by a wash in 0.1×SSC at 50° C. for 45 min before autoradiography. Alternatively, exemplary conditions of high stringency are as follows: e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & sons, Inc., New York, at p. 2.10.3). Other conditions of high stringency which may be used are well known in the art. In general, for probes between 14 and 70 nucleotides in length the melting temperature (TM) is calculated using the formula: Tm(° C.)=81.5+16.6(log [monovalent cations (molar)])+0.41 (% G+C)−(500/N) where N is the length of the probe. If the hybridization is carried out in a solution containing formamide, the melting temperature is calculated using the equation Tm(° C.)=81.5+16.6(log[monovalent cations (molar)])+0.41 (% G+C)-(0.61% formamide)−(500/N) where N is the length of the probe. In general, hybridization is carried out at about 20-25 degrees below Tm (for DNA-DNA hybrids) or 10-15 degrees below Tm (for RNA-DNA hybrids).

The tissue specific promoter such as the PC-1 regulatory sequence comprising nucleotides 1-322 of SEQ ID NO:1, or transcriptionally functional fragments thereof, is preferably derived from a mammalian organism. In one embodiment the PC-1 regulatory sequence comprising nucleotides 1-322 of SEQ ID NO:1 may be human, mouse or rat-derived. In another embodiment, as the PC-1 regulatory sequence comprising nucleotides 1-322 of SEQ ID NO:1, or transcriptionally functional fragments thereof. Screening procedures which rely on nucleic acid hybridization make it possible to isolate gene sequences from various organisms. The isolated polynucleotide sequence disclosed herein, or fragments thereof, may be labeled and used to screen a cDNA library constructed from mRNA obtained from appropriate cells or tissues (e.g., prostate tissue) derived from the organism of interest. The hybridization conditions used should be of a lower stringency when the cDNA library is derived from an organism different from the type of organism from which the labeled sequence was derived. Low stringency conditions are well know to those of skill in the art, and will vary depending on the specific organisms from which the library and the labeled sequence are derived. For guidance regarding such conditions see, for example, Sambrook et al., 1989, Molecular Cloning, A Laboratory Manual, Second Edition, Cold Spring Harbor Press, N.Y., and Ausabel et al., 1989, Current Protocols in Molecular Biology, Green Publishing Associates and Wiley Interscience, N.Y., each of which is incorporated herein by reference in its entirety.

Further, mammalian PC-1 transcriptional regulatory sequence homologues may be isolated from, for example, bovine or other non-human nucleic acid, by performing polymerase chain reaction (PCR) amplification using two primer pools designed on the basis of the nucleotide sequence of as the PC-1 regulatory sequence comprising nucleotides 1-322 of SEQ ID NO:1 disclosed herein. The template for the reaction may be cDNA obtained by reverse transcription of the mRNA prepared from, for example, bovine or other non-human cell lines, or tissue known to express PC-1. For guidance regarding such conditions, see, e.g., Innis et al. (Eds.) 1995, PCR Strategies, Academic Press Inc., San Diego; and Erlich (ed) 1992, PCR Technology, Oxford University Press, New York, each of which is incorporated herein by reference in its entirety.

Promoter sequences within the 5′ non-coding regions of the tissue specific promoter such as the PC-1 regulatory sequence comprising nucleotides 1-322 of SEQ ID NO:1 may be further defined by constructing nested 5′ and/or 3′ deletions using conventional techniques such as exonuclease III or appropriate restriction endonuclease digestion. The resulting deletion fragments can be inserted into the promoter reporter vector to determine whether the deletion has reduced or obliterated promoter activity, such as described, for example, by Coles et al. (Hum. Mol. Genet., 7:791-800, 1998). In this way, the boundaries of the promoters may be defined. If desired, potential individual regulatory sites within the promoter may be identified using site directed mutagenesis or linker scanning to obliterate potential transcription factor binding sites within the promoter individually or in combination. The effects of these mutations on transcription levels may be determined by inserting the mutations into cloning sites in promoter reporter vectors. These types of assays are well known to those skilled in the art (WO 97/17359, U.S. Pat. No. 5,374,544, EP 582 796, U.S. Pat. No. 5,698,389, U.S. Pat. No. 5,643,746, U.S. Pat. No. 5,502,176, and U.S. Pat. No. 5,266,488).

The tissue specific promoter such as the PC-1 regulatory sequence comprising nucleotides 1-322 of SEQ ID NO:1, or transcriptionally functional fragments thereof, and the fragments and probes described herein which serve to identify the tissue specific promoter such the PC-1 regulatory sequence comprising nucleotides 1-322 of SEQ ID NO:1, or transcriptionally functional fragments thereof, may be produced by recombinant DNA technology using techniques well known in the art. Methods which are well known to those skilled in the art can be used to construct these sequences, either in isolated form or contained in expression vectors. These methods include, for example, in vitro recombinant DNA techniques, synthetic techniques and in vivo genetic recombination. See, e.g., the techniques described in Sambrook et al., 1989, supra, and Ausabel et al., 1989, supra; also see the techniques described in “Oligonucleotide Synthesis”, 1984, Gait M. J. ed., IRL Press, Oxford, which is incorporated herein by reference in its entirety.

Alterations in the regulatory sequences can be generated using a variety of chemical and enzymatic methods which are well known to those skilled in the art. For example, regions of the sequences defined by restriction sites can be deleted. Oligonucleotide-directed mutagenesis can be employed to alter the sequence in a defined way and/or to introduce restriction sites in specific regions within the sequence. Additionally, deletion mutants can be generated using DNA nucleases such as Bal31, ExoIII, or S1 nuclease. Progressively larger deletions in the regulatory sequences are generated by incubating the DNA with nucleases for increased periods of time (see, e.g., Ausubel et al., 1989, supra).

The altered sequences are evaluated for their ability to direct expression of heterologous coding sequences in appropriate host cells. It is within the scope of the present invention that any altered regulatory sequences which retain their ability to direct expression of a coding sequence be incorporated into recombinant expression vectors for further use.

5.5 Analysis of PC-1-Specific Promoter Activity

The tissue specific promoter such as the PC-1 regulatory sequence comprising nucleotides 1-322 of SEQ ID NO:1 shows selective tissue and cell-type specificity; i.e., it induces gene expression in cells including, for example, and without limitation, prostate cancer cells, prostate stromal cells, breast cancer cells, renal cells, bladder cells, and cells of the endometrium. Thus, the PC-1 regulatory sequence comprising nucleotides 1-322 of SEQ ID NO:1, and transcriptionally active fragments thereof, of the present invention may be used to induce expression of a heterologous coding sequence in prostate cancer cells, prostate stromal cells, breast cancer cells, renal cells, bladder cells, and cells of the endometrium. In one embodiment, the present invention provides for the use of the tissue specific promoter such as the PC-1 regulatory sequence comprising nucleotides 1-322 of SEQ ID NO:1 to achieve tissue specific expression of a target gene. The activity and the specificity of the tissue specific promoter such as the PC-1 regulatory sequence comprising nucleotides 1-322 of SEQ ID NO:1 can further be assessed by monitoring the expression level of a detectable polynucleotide operably associated with the tissue specific promoter such as the PC-1 regulatory sequence comprising nucleotides 1-322 of SEQ ID NO:1 in different types of cells and tissues. As discussed hereinbelow, the detectable polynucleotide may be either a polynucleotide that specifically hybridizes with a predefined oligonucleotide probe, or a polynucleotide encoding a detectable protein.

5.6 The PC-1 Transcriptional Regulatory Sequence Driven Reporter Constructs

The PC-1 transcriptional regulatory sequences according to the invention may also be advantageously part of a recombinant expression vector that may be used to express a coding sequence, or reporter gene, in a desired host cell or host organism. The PC-1 transcriptional regulatory sequence of the present invention, and transcriptionally active fragments thereof, may be used to direct the expression of a heterologous coding sequence. In particular, the present invention encompasses mammalian PC-1 transcriptional regulatory sequences. In accordance with the present invention, transcriptionally active fragments of the PC-1 transcriptional regulatory sequence encompass those fragments of the region which are of sufficient length to promote transcription of a reporter coding sequence to which the fragment is operatively linked.

A variety of reporter gene sequences well known to those of skill in the art can be utilized, including, but not limited to, genes encoding fluorescent proteins such as green fluorescent protein (GFP), enzymes (e.g. CAT, beta-galactosidase, luciferase) or antigenic markers. For convenience, enzymatic reporters and light-emitting reporters analyzed by colorometric or fluorometric assays are preferred for the screening assays of the invention.

In one embodiment, for example, a bioluminescent, chemiluminescent or fluorescent protein can be used as a light-emitting reporter in the invention. Types of light-emitting reporters, which do not require substrates or cofactors, include, but are not limited to the wild-type green fluorescent protein (GFP) of Victoria aequoria (Chalfie et al., 1994, Science 263:802-805), and modified GFPs (Heim et al., 1995, Nature 373:663-4; PCT publication WO 96/23810). Transcription and translation of this type of reporter gene leads to the accumulation of the fluorescent protein in test cells, which can be measured by a fluorimeter, or a flow cytometer, for example, by methods that are well known in the art (see, e.g., Lackowicz, 1983, Principles of Fluorescence Spectroscopy, Plenum Press, New York).

Another type of reporter gene that may be used are enzymes that require cofactor(s) to emit light, including but not limited to, Renilla luciferase. Other sources of luciferase also are well known in the art, including, but not limited to, the bacterial luciferase (luxAB gene product) of Vibrio harveyi (Karp, 1989, Biochim. Biophys. Acta 1007:84-90; Stewart et al. 1992, J. Gen. Microbiol, 138:1289-1300), and the luciferase from firefly, Photinus pyralis (De Wet et al. 1987, Mol. Cell. Biol. 7:725-737), which can be assayed by light production (Miyamoto et al., 1987, J. Bacteriol. 169:247-253; Loessner et al. 1996, Environ. Microbiol. 62:1133-1140; and Schultz & Yarus, 1990, J. Bacteriol. 172:595-602).

Reporter genes that can be analyzed using colorimetric analysis include, but are not limited to, P-galactosidase (Nolan et al. 1988, Proc. Natl. Acad. Sci. USA 85:2603-07), P-glucuronidase (Roberts et al. 1989, Curr. Genet. 15:177-180), luciferase (Miyamoto et al., 1987, J. Bacteriol. 169:247-253), or β-lactamase. In one embodiment, the reporter gene sequence comprises a nucleotide sequence which encodes a LacZ gene product, β-galactosidase. The enzyme is very stable and has a broad specificity so as to allow the use of different histochemical, chromogenic or fluorogenic substrates, such as, but not limited to, 5-bromo-4-chloro-3-indoyl-β-D-galactoside (X-gal), lactose 2,3,5-triphenyl-2H-tetrazolium (lactose-tetrazolium) and fluorescein galactopyranoside (see Nolan et al., 1988, supra).

In another embodiment, the product of the E. coli β-glucuronidase gene (GUS) can be used as a reporter gene (Roberts et al. 1989, Curr. Genet. 15:177-180). GUS activity can be detected by various histochemical and fluorogenic substrates, such as X-glucuronide (Xgluc) and 4-methylumbelliferyl glucuronide.

In addition to reporter gene sequences such as those described above, which provide convenient colorimetric responses, other reporter gene sequences, such as, for example, selectable reporter gene sequences, can routinely be employed. For example, the coding sequence for chloramphenicol acetyl transferase (CAT) can be utilized, leading to PC-1 transcriptional regulatory sequence-dependent expression of chloramphenicol resistant cell growth. The use of CAT and the advantages of a selectable reporter gene are well known to those skilled in the art (Eikmanns et al 1991, Gene 102:93-98). Other selectable reporter gene sequences also can be utilized and include, but are not limited to, gene sequences encoding polypeptides which confer zeocin (Hegedus et al. 1998, Gene 207:241-249) or kanamycin resistance (Friedrich & Soriano, 1991, Genes. Dev. 5:1513-1523).

Other reporter genes, such as toxic gene products, potentially toxic gene products, and antiproliferation or cytostatic gene products, also can be used. In another embodiment, the detectable reporter polynucleotide may be either a polynucleotide that specifically hybridizes with a predefined oligonucleotide probe, or a polynucleotide encoding a detectable protein. This type of assay is well known to those skilled in the art (U.S. Pat. No. 5,502,176 and U.S. Pat. No. 5,266,488).

PC-1 transcriptional regulatory sequence driven reporter constructs can be constructed according to standard recombinant DNA techniques (see, e.g., Methods in Enzymology, 1987, volume 154, Academic Press; Sambrook et al. 1989, Molecular Cloning—A Laboratory Manual, 2nd Edition, Cold Spring Harbor Press, New York; and Ausubel et al. Current Protocols in Molecular Biology, Greene Publishing Associates and Wiley Interscience, New York, each of which is incorporated herein by reference in its entirety).

Methods for assaying promoter activity are well-known to those skilled in the art (see, e.g., Sambrook et al., Molecular Cloning A Laboratory Manual, Cold Spring Harbor Laboratory, Cold Spring Harbor, N.Y., 1989). An example of a typical method that can be used involves a recombinant vector carrying a reporter gene and sequences from an PC-1 transcriptional regulatory sequence. Briefly, the expression of the reporter gene (for example, green fluorescent protein, luciferase, β-galactosidase or chloramphenicol acetyl transferase) is detected when placed under the control of a biologically active polynucleotide fragment. Genomic sequences located upstream of the first exon of the PC-1 gene comprising SEQ ID NO:1 may be cloned into any suitable promoter reporter vector. For example, a number of commercially available vectors can be engineered to insert the PC-1 transcriptional regulatory sequence of the invention for expression in mammalian host cells. Non-limiting examples of such vectors are pSAPBasic, pSEAP-Enhancer, pβgal-Basic, pβgal-Enhancer, or pEGFP-1 Promoter Reporter vectors (Clontech, Palo Alto, Calif.) or pGL2-basic or pGL3-basic promoterless luciferase reporter gene vector (Promega, Madison, Wis.). Each of these promoter reporter vectors include multiple cloning sites positioned upstream of a reporter gene encoding a readily assayable protein such as secreted alkaline phosphatase, green fluorescent protein, luciferase or β-galactosidase. The PC-1 transcriptional regulatory sequences of the PC-1 gene are inserted into the cloning sites upstream of the reporter gene in both orientations and introduced into an appropriate host cell. The level of reporter protein is assayed and compared to the level obtained with a vector lacking an insert in the cloning site. The presence of an elevated expression level in the vector containing the insert with respect the control vector indicates the presence of a promoter or a functional fragment thereof in the insert.

Expression vectors that comprise a PC-1 transcriptional regulatory sequence may further contain a gene encoding a selectable marker. A number of selection systems may be used, including but not limited to, the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026) and adenine phosphoribosyltransferase (Lowy et al., 1980, Cell 22:817) genes, which can be employed in tk⁻, hgprt⁻ or aprt⁻ cells, respectively. Also, antimetabolite resistance can be used as the basis of selection for dhfr, which confers resistance to methotrexate (Wigler et al., 1980, Proc. Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes. Additional selectable genes include trpB, which allows cells to utilize indole in place of tryptophan; hisD, which allows cells to utilize histinol in place of histidine (Hartman & Mulligan, 1988, Proc. Natl. Acad. Sci. USA 85:8047); ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.) and glutamine synthetase (Bebbington et al., 1992, Biotech 10: 169).

5.7 Characterization of Transcriptionally Active Regulatory Fragments of the PC-1 Transcriptional Regulatory Sequence

A fusion construct comprising a PC-1 transcriptional regulatory sequence, or a fragment thereof, can be assayed for transcriptional activity. As a first step in promoter analysis of the PC-1 transcriptional regulatory sequence, the transcriptional start point (+1 site) of the PC-1 transcriptional regulatory sequence under study has to be determined using primer extension assay and/or RNAase protection assay, following standard methods (Sambrook et al., 1989, Molecular Cloning: A Laboratory Manual, Cold Spring Harbor, Cold Spring Harbor Press). The DNA sequence upstream of the +1 site is generally considered as the promoter region responsible for gene regulation. However, downstream sequences, including sequences within introns, also may be involved in gene regulation. To begin testing for promoter activity, a −3 kb to +3 kb region (where +1 is the transcriptional start point) may be cloned upstream of the reporter gene coding region. Two or more additional reporter gene constructs also may be made which contain 5′ and/or 3′ truncated versions of the regulatory region to aid in identification of the region responsible for prostate-specific expression. The choice of the type of reporter gene is made based on the application.

In a preferred embodiment, a GFP reporter gene construct is used. The application of green fluorescent protein (GFP) as a reporter is particularly useful in the study of prostate-specific gene promoters. A major advantage of using GFP as a reporter lies in the fact that GFP can be detected in freshly isolated prostate cells without the need for substrates.

In another embodiment of the invention, a Lac Z reporter construct is used. The Lac Z gene product, β-galactosidase, is extremely stable and has a broad specificity so as to allow the use of different histochemical, chromogenic or fluorogenic substrates, such as, but not limited to, 5-bromo-4-chloro-3-indoyl-β-D-galactoside (X-gal), lactose 2,3,5-triphenyl-2H-tetrazolium (lactose-tetrazolium) and fluorescein galactopyranoside (see Nolan et al., 1988, supra).

For promoter analysis in transgenic mice, GFP that has been optimized for expression in mammalian cells is preferred. The promoterless cloning vector pEGFP1 (Clontech, Palo Alto, Calif.) encodes a red shifted variant of the wild-type GFP which has been optimized for brighter fluorescence and higher expression in mammalian cells (Cormack et al., 1996, Gene 173:33; Haas et al., 1996, Curr. Biol. 6: 315). Moreover, since the maximal excitation peak of this enhanced GFP (EGFP) is at 488 nm, commonly used filter sets such as fluorescein isothiocyanate (FITC) optics which illuminate at 450-500 nm can be used to visualize GFP fluorescence. pEGFP1 proved to be useful as a reporter vector for promoter analysis in transgenic mice (Okabe et al, 1997, FEBS Lett. 407: 313). In an alternate embodiment, transgenic mice containing transgenes with a PC-1 transcriptional regulatory sequence upstream of the Lac Z or luciferase reporter genes are utilized.

Putative promoter fragments can be prepared (usually from a parent phage clone containing 8-10 kb genomic DNA including the promoter region) for cloning using methods known in the art. However, the feasibility of this method depends on the availability of proper restriction endonuclease sites in the regulatory fragment. In a preferred embodiment, the required promoter fragment is amplified by polymerase chain reaction (PCR; Saiki et al., 1988, Science 239:487) using oligonucleotide primers bearing the appropriate sites for restriction endonuclease cleavage. The sequence necessary for restriction cleavage is included at the 5′ end of the forward and reverse primers which flank the regulatory fragment to be amplified. After PCR amplification, the appropriate ends are generated by restriction digestion of the PCR product. The promoter fragments, generated by either method, are then ligated into the multiple cloning site of the reporter vector following standard cloning procedures (Sambrook et al., 1989, supra). It is recommended that the DNA sequence of the PCR generated promoter fragments in the constructs be verified prior to generation of transgenic animals. The resulting reporter gene construct will contain the putative promoter fragment located upstream of the reporter gene open reading frame, e.g., GFP or Lac Z cDNA.

5.8 PC-1 Transcriptional Regulatory Sequence Analysis Using Transgenic Mice

The mammalian PC-1 transcriptional regulatory sequence can be used to direct expression of, inter alia, a reporter coding sequence, a homologous gene or a heterologous gene in transgenic animals. Animals of any species, including, but not limited to, mice, rats, rabbits, guinea pigs, pigs, micro-pigs, goats, sheep, and non-human primates, e.g., baboons, monkeys and chimpanzees may be used to generate transgenic animals. The term “transgenic,” as used herein, refers to non-human animals expressing PC-1 transcriptional regulatory sequence from a different species (e.g., mice expressing PC-1 transcriptional regulatory sequence from either the rat or human PC-1 gene), as well as animals that have been genetically engineered to over-express endogenous (i.e., same species) PC-1 transcriptional regulatory sequence or animals that have been genetically engineered to knock-out specific sequences.

In one embodiment, the present invention provides for transgenic animals that carry a transgene such as a reporter gene under the control of the PC-1 transcriptional regulatory sequence or transcriptionally active fragments thereof in all their cells, as well as animals that carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (1992, Proc. Natl. Acad. Sci. USA 89:6232-6236). When it is desired that the transgene be integrated into the chromosomal site of the endogenous corresponding gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene.

Any technique known in the art may be used to introduce a transgene under the control of the PC-1 transcriptional regulatory sequence into animals to produce the founder lines of transgenic animals. Such techniques include, but are not limited to, pronuclear microinjection (Hoppe & Wagner, 1989, U.S. Pat. No. 4,873,191); nuclear transfer into enucleated oocytes of nuclei from cultured embryonic, fetal or adult cells induced to quiescence (Campbell et al., 1996, Nature 380:64-66; Wilmut et al., Nature 385:810-813); retrovirus gene transfer into germ lines (Van der Putten et al., 1985, Proc. Natl. Acad. Sci., USA 82:6148-6152); gene targeting in embryonic stem cells (Thompson et al., 1989, Cell 65:313-321); electroporation of embryos (Lo, 1983, Mol. Cell. Biol. 31:1803-1814); and sperm-mediated gene transfer (Lavitrano et al., 1989, Cell 57:717-723; see, Gordon, 1989, Transgenic Animals, Intl. Rev. Cytol. 115:171-229).

5.9 Screening Assays

Compounds that interfere with the abnormal function and/or growth of prostate cancer cells, prostate stromal cells, breast cancer cells, renal cells, bladder cells, and cells of the endometrium can provide therapies targeting defects in prostate-related disorders. Such compounds may be used to interfere with the onset or the progression of prostate-related disorders, prostate cancer, brain cancer, ovarian cancer, thyroid cancer, tumors, lung cancer, renal carcinoma, bladder cancer, or breast cancer. Compounds that stimulate or inhibit promoter activity may be used to ameliorate symptoms of prostate-related disorders.

Transgenic animals or cells containing a PC-1 transcriptional regulatory sequence, or fragment thereof, operably linked to a reporter gene, can be used as systems for the screening of agents that modulate PC-1 transcriptional regulatory sequence activity. Such agents that modulate PC-1 transcriptional regulatory sequence activity can then be used to develop new methods of treatment of prostate cancer cells, prostate stromal cells, breast cancer cells, renal cells, bladder cells, and cells of the endometrium. In addition, PC-1 transcriptional regulatory sequence containing transgenic mice provide an experimental model both in vivo and in vitro to develop new methods of treating prostate-related disorders, prostate cancer, brain cancer, ovarian cancer, thyroid cancer, tumors, lung cancer, renal carcinoma, bladder cancer, or breast cancer by targeting drugs to cause arrest in the progression of such disorders.

The present invention encompasses screening assays designed to identify compounds that modulate activity of the PC-1 transcriptional regulatory sequence and/or the PC-1 gene. The present invention encompasses in vitro and cell-based assays, as well as in vivo assays in transgenic animals. As described hereinbelow, compounds to be tested may include, but are not limited to, oligonucleotides, peptides, proteins, small organic or inorganic compounds, antibodies, etc.

Examples of compounds may include, but are not limited to, peptides, such as, for example, soluble peptides, including, but not limited to, Ig-tailed fusion peptides, and members of random peptide libraries; (see, e.g., Lam, et al., 1991, Nature 354:82-84; Houghten, et al., 1991, Nature 354:84-86), and combinatorial chemistry-derived molecular library made of D- and/or L-configuration amino acids, phosphopeptides (including, but not limited to members of random or partially degenerate, directed phosphopeptide libraries; see, e.g., Songyang, et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

Such compounds may further comprise compounds, in particular drugs or members of classes or families of drugs, known to ameliorate the symptoms of a prostate-related disorder.

Such compounds include, but are not limited to, families of antidepressants such as lithium salts, carbamazepine, valproic acid, lysergic acid diethylamide (LSD), p-chlorophenylalanine, p-propyldopacetamide dithiocarbamate derivatives e.g., FLA 63; anti-anxiety drugs, e.g., diazepam; monoamine oxidase (MAO) inhibitors, e.g., iproniazid, clorgyline, phenelzine and isocarboxazid; biogenic amine uptake blockers, e.g., tricyclic antidepressants such as desipramine, imipramine and amitriptyline; serotonin reuptake inhibitors e.g., fluoxetine; antipsychotic drugs such as phenothiazine derivatives (e.g., chlorpromazine (thorazine) and trifluopromazine)), butyrophenones (e.g., haloperidol (Haldol)), thioxanthene derivatives (e.g., chlorprothixene), and dibenzodiazepines (e.g., clozapine); benzodiazepines; dopaminergic agonists and antagonists e.g., L-DOPA, cocaine, amphetamine, α-methyl-tyrosine, reserpine, tetrabenazine, benzotropine, pargyline; noradrenergic agonists and antagonists e.g., clonidine, phenoxybenzamine, phentolamine, tropolone; nitrovasodilators (e.g., nitroglycerine, nitroprusside as well as NO synthase enzymes); and growth factors (e.g., VEGF, FGF, angiopoetins and endostatin).

In one preferred embodiment, primary cultures of germ cells containing a mammalian PC-1 transcriptional regulatory sequence operatively linked to a heterologous gene are used to develop assay systems to screen for compounds which can inhibit or enhance sequence-specific DNA-protein interactions. Such methods comprise contacting a compound to a cell that expresses a gene under the control of a PC-1 transcriptional regulatory sequence, or a transcriptionally active fragment thereof, measuring the level of the gene expression or gene product activity and comparing this level to the level of gene expression or gene product activity produced by the cell in the absence of the compound, such that if the level obtained in the presence of the compound differs from that obtained in its absence, a compound capable of modulating the expression of the mammalian PC-1 transcriptional regulatory sequence has been identified. Alterations in gene expression levels may be by any number of methods known to those of skill in the art e.g., by assaying for reporter gene activity, assaying cell lysates for mRNA transcripts, e.g. by Northern analysis or using other methods known in the art for assaying for gene products expressed by the cell.

Once a compound has been identified that inhibits or enhances PC-1 transcriptional regulatory sequence activity, it may then be tested in an animal-based assay to determine if the compound exhibits the ability to act as a drug to ameliorate and/or prevent symptoms of prostate cancer, brain cancer, ovarian cancer, thyroid cancer, tumors, lung cancer, renal carcinoma, bladder cancer, or breast cancer and/or prevent the proliferation of cancer cells, including, for example, without limitation, prostate cancer cells, prostate stromal cells, breast cancer cells, renal carcinoma cells, bladder carcinoma cells, and endometrial carcinoma cells.

The invention is illustrated by way of the following non-limiting examples.

5.10 Identification and Characterization of the PC-1 Gene

5.11 Materials and Methods

5.11.1 Microarray Analysis

Total RNA was isolated from LNCaP and C4-2 cells using RNAzol obtained from Biotech Laboratories, Inc. (Houston, Tex.) according to the manufacturer's instructions. Microarray probe construction, microarray hybridization, and analysis were described in detail elsewhere.

5.11.2 Cell Lines and Cell Cultures

Parental LNCaP and its derivative cell lines, C4, C4-2, and C4-2B, were described previously. ARCaP cells, a highly invasive prostate cancer cell line, were generated by our lab from the ascites fluid of a man with Al and aggressive human prostate cancer. All other cell lines were obtained from ATCC and were cultured in TB containing 5% FBS, as described previously. To test the effect of androgen in affecting PC-1 gene expression in prostate cancer cell lines, cells were exposed to a synthetic androgen methyltrioenolone (R1881) at a concentration of 0.1, 1, and 10 nM. To assess the specificity of androgen induction of PC-1 gene expression, some of the cells exposed to 10 nM of R1881 were also treated with an antiandrogen Casodex, 10 μM. The culture was allowed to continue for two days and total cellular RNA was isolated and subjected to RT-PCR and Northern blot analysis for the expression of PC-1 gene.

5.11.3 Northern Blot Hybridization

Ten micrograms of total RNA was electrophoresed from 1% agarose denaturing gel and transferred to a Zeta Probe-charged nylon membrane and RNA was UV cross-linked to the membrane. Blots were hybridized overnight at 65° in Rapid Hybr solution with DNA probes labeled with P32 dCTP by random priming method using the random primer DNA labeling kits obtained from Amersham Pharmacia Biotech according to the manufacturer's protocol. In some studies, Northern hybridization was conducted against human multi-tissue cDNA blots obtained from Clontech (Palo Alto, Calif.).

5.11.4 Reverse Transcription Polymerase Chain Reaction

First strain cDNA was synthesized from 1 μg of total RNA with a hexamer random primer obtained from Perkin-Elmer using a MMLV reverse transcriptase obtained from Life Technologies, Inc. (Grand Island, N.Y.) according to the manufacturer's instructions. cDNAs obtained were subjected to Taq DNA polymerase PCR reaction under the condition of 94° for 1 minute, 60° for 1 minute, 72° for 45 seconds, or 94° for 1 minute, 53° for 1 minute, and 72° for 1 minute for 30 cycles, and an extension of 72° for 10 minutes. For obtaining the complete sequence of the PC-1 gene, 5′-RACE and 3′-RACE were performed using the Clontech Smart TM RACE cDNA amplification kit (Palo Alto, Calif.) according to the manufacturer's instructions. Gene-specific primers (GSP) were designed to be used for 5′- and 3′-RACE cDNA amplification procedure. The sequence of GSP-1 is 5′-AACACCAGGTGGTGAATAGAGCCCCTCC-3′, and GSP-2 is 5′-CATGGCTGTCTAATCGCCTGATATCAGCC-3′. The 5′- and 3′-RACE PCR products share a 247 bp EST sequence overlapping region. The PCR. products were evaluated on a 1% agarose gel and confirmed by Southern blot analysis. PCR products were gel purified and TA cloned into PCR 2.1-Topo vector obtained from Invitrogen and sequenced using M 13 reverse and forward primers.

5.12 Results and Discussion

A total of 1,600 distinct cDNAs from the prostate were used as a template for microarray using cDNA prepared from LNCaP and C4-2 cell lines. Sixteen cDNAs were identified with 2-6 fold higher expression in C4-2 than LNCaP cells. Nine cDNA clones were selected that revealed higher fold expression in C4-2 cells as targets for Northern blot analysis. Results show that one of the clones, 5B-10, was overexpressed at least 5 fold in C4-2 cells than that of the parental LNCaP. The EST used to hybridize 5B-10 gene was subsequently sequenced and was found to share a high degree of homology with tvo known proteins, D52 and N8, with gene bank accession numbers 4827037 and #582081, respectively. Because of areas of dissimilarity with clone 5B-10 and D52/N8, five prime and three prime RACE was performed to determine the full length of the PC-1 cDNA.

After appropriate 5′- and 3′-RACE using a Clontech kit (Palo Alto, Calif.), a 2.6 Kb full length cDNA including the cDNA of PC-1 gene, a polyadenylation signal, and a poly-A tail was obtainbed. BLAST advanced sequence comparison search revealed that the 2.6 Kb full length cDNA sequence contained three distinct regions: 1) 3′-end of PC-1 cDNA sequences shared 97% homology to an ESD sequence (48-355 bp); 2) a 157 bp sequence shared no homology with any known genes; and, 3) the 2.1 Kb at the three prime end was found to be identical to a partial sequence of known genes such as D52 (sequence from 120-2178 bp) and N8. The Entrez ORF Finder revealed that the full length cDNA of the PC-1 gene has an open reading frame presumably initiated by a methionine that is located at position 323 bp with a stop codon at position 997 bp. This predicts a protein of 24 kDa or 224 amino acids of a PC-1 gene. The PC-1 gene shares 82% sequence homology at the nucleotide level to the N8 gene. The difference between PC-1 and D52/N8 is at the 5′-region. The PC-1 gene has a different and longer, 323 bp, five prime untranslated region and an extra 46 amino acids at the N-terminal of the protein within the coding sequence. The PC-1 gene shares marked homology to D52/N8, N8L, and mD52 for mouse, CSPP-28 from rabbit, and R-10 from quail. The protein sequence of this family member is highly conserved between species. Using Northern blot analysis, by probing a Northern filter membrane with two sets of probes, a PC-1 gene specific probe and a N8/D52 common sequence probe, with the PC-1 gene resulted in a confirmation of PC-1 gene which resided at 2.6 Kb, as opposed at D52/N8 which resides at 2.4 Kb. RT-PCR was performed to distinguish PC-1 and D52/N8 genes. A representative 1 Kb PC-1 gene and 700 bp D52/N8 gene were obtained following RT-PCR reaction. Using a similar technique, the PC-1 gene was identified in normal prostate, BPH, and prostate cancer tissues. Other than human prostate cell lines, a number of other tumor types such as breast, bladder, endometrium, renal cell carcinoma, and osteosarcoma cell line MG-63 were also found to expressed the PC-1 gene. Tissue distribution of the PC-1 gene was confirmed by both Northern blot and RT-PCR. Results of these studies indicate that PC-1 gene expression is specifically associated with normal prostate tissues with minor expression in colon- and kidney-derived tissues. Other human organs including heart, brain, placenta, lung, liver, skeletal muscle, pancreas, spleen, thymus, testes, ovaries, small intestines, and peripheral leukocytes were found to be devoid of PC-1 gene expression. The PC-1 gene was induced by androgen in androgen-sensitive prostate cancer cell lines such as LNCaP and C4-2. Androgen-induced PC-1 gene expression in these prostate cancer cell lines can be effectively terminated by the coadministration of an antiandrogen Casodex, suggesting that androgen receptors may be responsible for up-regulation of PC-1 gene expression in prostate cancer cell lines.

In summary, the present invention demonstrates for the first time that the PC-1 gene is a member of the D52/N8 gene family, and is differentially expressed in normal prostate tissues and benign and malignant prostate tissues. PC-1 gene expression appears to be androgen regulated and is mapped to chromosome 81q21, a region frequently amplified in human prostate cancer. Unlike the D52/N8 gene, PC-1 gene expression is up-regulated by androgen and appears to have a differential pattern of distribution in normal human tissues. Thus, the PC-1 gene may be used as a diagnostic, prognostic, and treatment target for therapy. Moreover, the promoter/regulatory region of the PC-1 gene can be used for basic and clinical investigations to improve the understanding of the basic mechanisms of gene expression, androgen regulation, and as a tool to deliver therapeutic genes to prostate cells for basic and therapeutic applications in disease states.

6. Analysis of PC-1 Gene Expression by Northern Blot Hybridization

6.1 Materials and Methods

Source of the human mRNA samples. A nylon membrane containing the Human Multiple Tissue Expression (MET™) Array was obtained from Clontech Laboratories, Inc. (Catalog # 7775-1). Arrangement of the human mRNA samples on the membrane is depicted in Table X.

Source of the cDNA probe. A 460 bp corresponding to 5′ end of the PC1 cDNA was cloned into pBluescript following RT-PCR amplification. This cDNA fragment contains the complete 5′ untranslated region and the first 90 bp coding sequence of the PC1 cDNA. To prepare a probe for hybridization to the array, this 460 bp insert was purified from the vector and labeled with random priming method, with a-³²P-dATP used in the reaction.

Hybridization and autoradiography. The ExpressHyb™ Hybridization Solution that was included in the Human Multiple Tissue Expression (MET™) Array system was used in the hybridization reaction. About 3′ 10⁸ cpm of the redioactive probe was added to the reaction. Hybridization reaction was performed at 65° C. overnight. Subsequently, hybridized membrane was washed three times in 1′ SSC, 0.1% SDS at room temperature, followed by washing twice in 0.1′ SSC, 0.1% SDS at 65° C. for 20 minutes each. The membrane was then exposed to X-OMAT film (Kodak) at −80° C. for 72 hours.

6.2 Results and Conclusion

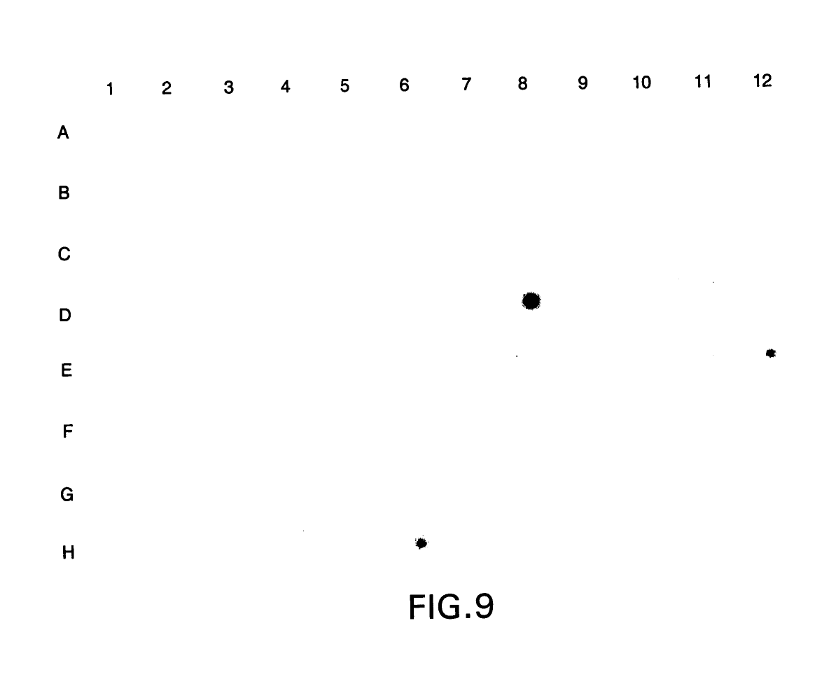

The results of the hybridization are shown in FIG. 9 This study shows that the human PC1 gene is predominantly expressed in prostate tissue (array coordinate E8)(Table 1). Meanwhile, this gene is expressed in a less degree in human gastrointestinal system. It is estimated that the expression of PC1 is at least 20 times higher in prostate than in the stomach (array coordinate B5). On the other hand, the expression of PC1 in other human tissues included in this array is minimal.

| | 1 | 2 | 3 | 4 | 5 | 6 | 7 | 8 | 9 | 10 | 11 | 12 |
|---|---|---|---|---|---|---|---|---|---|---|---|---|
| A | whole brain | cerebellum | substanita | heart | esophagus | colon | kidney | lung | liver | HL-60 | fetal | yeast |
| | | left | nigra | | | transverse | | | | leukemia | brain | total |
| | | | | | | | | | | | | RNA |
| B | cerebral | cerebellum | accumbens | aorta | stomach | colon | skeletal | placenta | pancreas | HeLa S3 | fetal | yeast |
| | cortex | right | nucleus | | | descending | muscle | | | | heart | tRNA |
| C | frontal | corpus | thalamus | atrium | duodenum | rectum | spleen | bladder | adrenal | K-562 | fetal | E coli |
| | lobe | callosum | | left | | | | | gland | leukemia | kidney | rRNA |
| D | parietal | amygdala | pituitary | atrium | jejunum | | thymus | uterus | thyroid | MOLT-4 | fetal | E coli |
| | lobe | | gland | right | | | | gland | leukemia | liver | | DNA |
| E | occipital | caudate | spinal | ventricle | ileum | | peripheral | prostate | salivary | Burkitt's | fetal | Poly |
| | lobe | nucleus | cord | left | | | Blood | | gland | lymphoma | thymus | r(A) |
| | | | | | | | leukocyte | | | Raji | | |
| F | temporal | hippocampus | | ventricle | ilocecum | | lymph | testis | mammary | Burkitt's | | human |
| | lobe | | | right | | | node | | gland | lymphoma | | Cₒt-l |
| | | | | | | | | | | daudi | | DNA |
| G | paracentral | medulla | | inter- | appendix | | bone | ovary | | SW480 | fetal | human |
| | gyrus of | oblongata | | ventricular | | | marrow | | | colorectal | lung | DNA |
| | cerebral | | | septum | | | | | | adeno- | | 100 ng |
| | cortex | | | | | | | | | carcinoma | | |
| H | pons | putamen | | apex | colon | | trachea | | | A549 lung | | human |
| | | | | | ascending | | | | | carcinoma | | DNA |
| | | | | | | | | | | | | 500 ng |

What is claimed is:

1. An isolated nucleic acid molecule comprising a nucleotide sequence that encodes a novel prostate specific protein comprising amino acid residues 1-224, 1-46 or 47-224 of the amino acid sequence depicted in FIG. 2.

2. An isolated nucleic acid molecule comprising nucleic acid residues 1-2552, 1-322, 323-461, 1-461, 323-997 or 323-2552 of the nucleic acid sequence depicted in FIG. 2.

3. An isolated nucleic acid molecule comprising a complement of the nucleic acid molecule of claim 1.

4. An isolated nucleic acid molecule which hybridizes to the nucleic acid molecule as in one of claims 1-3 under highly stringent conditions.

5. An isolated nucleic acid molecule which hybridizes to the nucleic acid molecule as in one of claims 1-3 under moderately stringent conditions.

6. A vector comprising the nucleic acid molecule of claim 4.

7. An expression vector comprising the nucleic acid molecule of claim 4 operatively associated with a regulatory nucleic acid controlling the expression of the nucleic acid in a host cell.

8. A host cell genetically engineered to express the nucleic acid molecule of claim 4.

9. A host cell genetically engineered to express the nucleic acid molecule of claim 4 operatively associated with a regulatory nucleic acid controlling the expression of the nucleic acid in the host cell.

10. A transgenic, non-human animal, which has been genetically engineered to contain a transgene comprising the nucleic acid molecule of claim 4.

11. An isolated polypeptide comprising amino acid residues 1-224, 1-46 or 47-224 of the amino acid sequence depicted in FIG. 2.

12. An isolated polypeptide comprising an amino acid sequence encoded by the isolated nucleic acid molecule of claim 4.

13. An antibody which binds to an isolated polypeptide comprising an amino acid sequence encoded by the isolated nucleic acid molecule of claim 4.

14. A method for identifying a compound which modulates expression of a novel prostate specific protein (PC-1) comprising:
(a) contacting a test compound to a cell that expresses a PC-1;
(b) measuring a level of the PC-1 expression in the cell; and
(c) comparing the level of the PC-1 expression in the cell in the presence of the test compound to a level of the PC-1 expression in the cell in the absence of the test compound;
wherein, if the level of the PC-1 expression in the cell in the presence of the test compound differs from the level of expression of the PC-1 in the cell in the absence of the test compound, a compound that modulates expression of the PC-1 is identified.

15. A method for transferring a novel prostate specific protein (PC-1) in a cell comprising contacting the cell with a nucleic acid comprising a PC-1 such that the PC-1 is transferred into the cell.

16. The method of claim 17 wherein the PC-1 is expressed in the cell.

17. A therapeutic agent comprising a prostate specific protein (“PC-1”) regulatory region, a delivery vector and a toxic, therapeutic and/or heterologous coding sequence.

18. The therapeutic agent of claim 17, further comprising a prodrug.

19. The therapeutic agent of claim 18, wherein said prodrug is selected from the group consisting of acyclovir (“ACV”) and gancyclovir (“GCV”).

20. The therapeutic agent of claim 17, further comprising an androgen.

21. The therapeutic agent of claim 17, wherein said PC-1 regulatory region comprises nucleic acid residues 1 to 322 depicted in FIG. 2.

22. The therapeutic agent of claim 17, wherein said delivery vector comprises a viral vector.

23. The therapeutic agent of claim 22, wherein said viral vector is an adenovirus.

24. The therapeutic agent of claim 17, wherein said delivery vector comprises a liposome.

25. The therapeutic agent of claim 17, wherein said toxic coding sequence is selected from the group consisting of thymidine kinase and cytosine deaminase.

26. The therapeutic agent of claim 17, wherein said therapeutic coding sequence is selected from the group consisting of growth factors, cytokines, therapeutic proteins, hormones and peptide fragments of hormones, inhibitors of cytokines, peptide growth and differentiation factors, interleukins, chemokines, interferons, colony stimulating factors and angiogenic factors.

27. The therapeutic agent of claim 17, wherein said heterologous coding sequence is a reporter gene.

28. A method for identifying a test compound capable of modulating prostate-specific gene expression comprising:
(a) measuring the level of expression of a reporter gene under the control of a PC-1 regulatory region, or a transcriptionally active fragment thereof, in the presence and absence of said test compound,
such that if the level obtained in the presence of the test compound differs from that obtained in its absence, then a compound which modulates prostate-specific gene expression is identified.

29. A pharmaceutical composition comprising the test compound identified by the method in claim 28.

30. A method for delivery of a toxic and/or therapeutic molecule comprising, introducing into prostate cells of a subject a vector comprising a PC-1 regulatory region sequence, or transcriptionally active fragment thereof, operatively linked to a heterologous nucleic acid which encodes said toxic and/or therapeutic molecule.

31. A method for treating and/or ameliorating an prostate-related disease or disorder comprising introducing into prostate cells of a subject a vector comprising a PC-1 regulatory region sequence, or transcriptionally active fragment thereof, operatively linked to a heterologous nucleic acid whose gene product is capable of treating and/or ameliorating said disease or disorder.

32. A method for treating and/or ameliorating an prostate-related cancer or other proliferative disorder comprising introducing into a cell of said cancer or other proliferative disorder of a subject a vector comprising a PC-1 regulatory region sequence, or transcriptionally active fragment thereof, a delivery vector and a toxic, therapeutic and/or heterologous coding sequence whose gene product is capable of killing said cell.

33. The method of claim 32 wherein said cancer or other proliferative disorder is selected from the group consisting of prostate cancer, breast cancer, renal cell carcinoma, bladder cancer, and endometrium carcinoma.

34. The method of claim 32 further comprising introducing a prodrug.

35. The method of claim 34 wherein said prodrug is selected from the group consisting of ACV and GCV.

36. The method of claim 32 wherein said introducing comprises administration via direct application, or systemic application via intravenous administration, intra-arterial administration, intra-tumoral administration, perfusion and oral administration.

37. The method of claim 32, wherein said PC-1 regulatory region sequence comprises nucleic acid residues 1-322 depicted in FIG. 2.

38. The method of claim 37, wherein said PC-1 regulatory region sequence comprises a nucleotide sequence which hybridizes under highly stringent conditions to the complement of nucleic acid residues 1-322 depicted in FIG. 2.

39. The method of claim 37, wherein said PC-1 regulatory region sequence comprises a nucleotide sequence which hybridizes under moderately stringent conditions to the complement of nucleic acid residues 1-322 depicted in FIG. 2.

40. A method for preventing or delaying a prostate-related disorder comprising introducing into prostate cells of a subject a vector comprising a PC-1 regulatory region sequence, or transcriptionally active fragment thereof, operatively linked to a heterologous nucleic acid which encodes a therapeutic coding sequence which is capable of preventing or delaying said disorder.

41. The method of claim 27, wherein said therapeutic coding sequence is selected from the group consisting of growth factors, cytokines, therapeutic proteins, hormones and peptide fragments of hormones, inhibitors of cytokines, peptide growth and differentiation factors, interleukins, chemokines, interferons, colony stimulating factors and angiogenic factors.

42. A method for modulating immune functions comprising administering a polynucleotide to an area where modulation of immune function is necessary, wherein said polynucleotide comprises a PC-1 regulatory region sequence, or transcriptionally active fragment thereof, a delivery vector and therapeutic coding sequence whose gene product is capable of modulating immune functions.

43. The method of claim 42, wherein said therapeutic coding sequence is selected from the group consisting of interferons alpha, beta or gamma; tumour necrosis factor; granulocyte-macrophage colony-stimulating factor (GM-CSF), granulocyte colony-stimulating factor (G-CSF), macrophage colony-stimulating factor (N-CSF), chemokines such as neutrophil activating protein NAP, macrophage chemoattractant and activating factor MCAF, RANTES, macrophage inflammatory peptides MIP-1a and MIP-1b, complement components and their receptors, accessory molecules such as 87.1, 87.2, ICAM-1.2 or 3 or cytokine receptors.


SEQUENCE LISTING (4 sequences)

SEQ ID NO: 1 - DNA, 2672 bp, Homo sapiens
gcccttctttgttgactgaggaaagctcgctccctgcccaggtttttcattgttgatcgaaattaacaccaggtggtgaatagagcccctcctaaggttgctcaggataaatcatttattaaataggtctgcttatcaggaggggcgtgaaggctcccaaaaggaaatgctggcacctgggcccagaagccagggcctctaactcctggggttgatttcttcagtgaagttgcaccctacaaagggaatatggccaaagcggcactcaactgaaggctgatatcaggcgattagacagccatgcattctgcgtttgtctggaatggattgtagagagatggacttatatgaggactaccagtccccgtttgattttgatgcaggagtgaacaaaagctatctctacttgtctcctagtggaaattcatctccacccggatcacctactcttcagaaatttggtctgctgagaacagacccagtccctgaggaaggagaagatgttgctgccacgatcagtgccacagagaccctctcggaagaggagcaggaagagctaagaagagaacttgcaaaggtagaagaagaaatccagactctgtctcaagtgttagcagcaaaagagaagcatctagcagagatcaagcggaaacttggaatcaattctctacaggaactaaaacagaacattgccaaagggtggcaagacgtgacagcaacatctgcttacaagaagacatctgaaaccttatcccaggctggacagaaggcctcagctgctttttcgtctgttggctcagtcatcaccaaaaagctggaagatgtaaaaaactccccaacttttaaatcatttgaagaaaaggtcgaaaacttaaagtctaaagtagggggaaccaagcctgctggtggtgattttggagaagtcttgaattcggctgcaaatgctagtgccaccaccacggagcctcttccagaaaagacacaggagagcctgtgagattcctacctttgttctgctacccactgccagatgctgcaagcgaggtccaagcacatcttgtcaacatgcattgccatgaatttctaccagatgtgcttttatttagctttacatattcctttgaccaaatagtttgtgggttaaacaaaatgaaaatatcttcacctctattcttgggaaacaccctttagtgtacatttatgttcctttatttaggaaacaccattataaaaacacttatagtaaatggggacattcactataatgatctaagaagctacagattgtcatagttgttttcctgctttacaaaattgctccagatctggaatgccagtttgacctttgtcttctataatatttcctttttttcccctctttgaatctctgtatatttgattcttaactaaaattgttctcttaaatattctgaatcctggtaattaaaagtttgggtgtattttctttacctccaaggaaagaactactagctacaaaaaatattttggaataagcattgttttggtataaggtacatattttggttgaagacaccagactgaagtaaacagctgtgcatccaatttattatagttttgtaagtaacaatatgtaatcaaatattttggaataagcattgttttggtataaggtacatattttggttgaagacaccagactgaagtaaacagctgtgcatccaatttattatagttttgtaagtaacaatatgtaatcaaacttctaggtgacttgagagtggaacctcctatatcattatttagcaccgtttgtgacagtaaccatttcagtgtattgtttattataccacttatatcaacttatttttcaccaggttaaaattttaatttctacaaaataacattctgaatcaagcacactgtatgttcagtaggttgaactatgaacactgtcatcaatgttcagttcaaaagcctgaaagtttagatctagaagctggtaaaaatgacaatatcaatcacattaggggaaccattgttgtcttcacttaatccatttagcactatttaaaataagcacaccaagttatatgactaatataacttgaaaattttttatactgaggggttggtgataactcttgaggatgtaatgcattaataaaaatcaactcatcattttctacttgttttcaatgtgttggaaactgtaaaatgatactgtagaacctgtctcctactttgaaaactgaatgtcagggctgagtgaatcaaagtgtctagacatatttgcatagaggccaaggtattctattctaataactgcttactcaacactaccaccttttccttatactgtatatgattatggcctacaatgttgtatttgttatttattaaattgtgattgttttattattgtttatgccaaatgttaactgccaagcttggagtgacctaaagcattttttaaaagcatggctagatttacttcagtataaattatcttatgaaaaccaaattttaaaagccacaggtgttgattgttataaaataacatgctgccattcttgattgctagagtttttgttagtactttggatgcaattaaaactatgtgctatcacatgtgaaaagcttaataaattccatctatcagtggaaaaaaaaaaaaaaaaaaaaaaaaaaaa

SEQ ID NO: 2 - protein, 224 aa, Homo sapiens
MDCREMDLYEDYQSPFDFDAGVNKSYLYLSPSGNSSPPGSPTLQKFGLLRTDPVPEEGEDVAATISATETLSEEEQEELRRELAKVEEEIQTLSQVLAAKEKHLAEIKRKLGINSLQELKQNIAKGWQDVTATSAYKKTSETLSQAGQKASAAFSSVGSVITKKLEDVKNSPTFKSFEEKVENLKSKVGGTKPAGGDFGEVLNSAANASATTTEPLPEKTQESL

SEQ ID NO: 3 - DNA, 28 bp, Homo sapiens
aacaccaggtggtgaatagagcccctcc

SEQ ID NO: 4 - DNA, 29 bp, Homo sapiens
catggctgtctaatcgcctgatatcagcc